(19) United States
(12) Patent Application Publication
(10) Pub. No.: US 2006/0205743 A1
(43) Pub. Date: Sep. 14, 2006

(54) Protein kinase inhibitors

(76) Inventors: Kenichiro Kataoka, Tokyo (JP); Tomomi Kosugi, Tokyo (JP); Minoru Imai, Tokyo (JP); Dale Robert Mitchell, Walden Essex (GB); Donald James Simpson, Walden Essex (GB); Naotaka Suzuki, Tokyo (JP); Yuko Yamakoshi, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/548,668

(22) PCT Filed: Mar. 11, 2004
(86) PCT No.: PCT/JP04/03247

§ 371 Date: Sep. 9, 2005

(30) Foreign Application Priority Data
Mar. 11, 2003 (GB) 0305559.7

(51) Int. Cl. A61K 31/519

(52) U.S. Cl. 514/259.3

(57) ABSTRACT

The Pyrazolo[1,5-a]pyrimidine derivatives represented by formula I and their pharmaceutically acceptable salts exhibit excellent kinase inhibiting activity. Drugs comprising the compounds as effective ingredients are therefore expected to be useful as therapeutic or prophylactic agents for a protein kinase mediated disorder in which kinase is implicated, such as inflammatory disease, autoimmune disease, destructive bone disorder, cancer and/or tumour growth.

### FIELD OF THE INVENTION

The present invention relates to the use of certain compounds in the inhibition of protein kinases, in particular inhibitors of mitogen-activated protein kinase (MAPK) family, more particularly serine/threonine kinases, mitogen-activated protein kinase-activated protein kinase 2 (MAPKAP-K2). Their use in medicine and particularly in the prevention and/or treatment of inflammatory and neurological disorders is described.

### BACKGROUND ART

Protein kinases are a family of enzymes that catalyse the phosphorylation of hydroxyl groups in proteins. Approximately 2% of the genes encoded by the human genome are predicted to encode protein kinases. The reversible phosphorylation of specific tyrosine, serine, or threonine residues on a target protein can dramatically alter its function in several ways including activating or inhibiting enzymatic activity; creating or blocking binding sites for other proteins; altering subcellular localisation or controlling protein stability. Consequently protein kinases are pivotal in the regulation of a wide variety of cellular processes, including metabolism, cell proliferation, differentiation and survival. Of the many different cellular functions known to require the actions of protein kinases, some represent targets for therapeutic intervention for certain disease states.

It is known that several diseases can arise from, or involve, aberrant protein kinase activity. In humans, protein tyrosine kinases are known to have a significant role in the development of many disease states including diabetes, cancer and have also been linked to a wide variety of congenital syndromes. Serine threonine kinases also represent a class of enzymes, inhibitors of which are likely to have relevance to the treatment of cancer, diabetes and a variety of inflammatory disorders. Modulation of protein kinase activity therefore represents an attractive area for the design of new therapeutic agents.

Three potential mechanisms for inhibition of protein kinases have been identified thus far. These include a pseudo-substrate mechanism, an adenine mimetic mechanism and the locking of the enzyme into an inactive conformation by using surfaces other than the active site. The majority of inhibitors identified/designed to date act at the ATP-binding site. Such ATP-competitive inhibitors have demonstrated selectivity by virtue of their ability to target the more poorly conserved areas of the ATP-binding site.

One of the principal mechanisms by which cellular regulation is effected is through the transduction of extracellular signals across the membrane that in turn modulate biochemical pathways within the cell. Protein phosphorylation represents one course by which intracellular signals are propagated from molecule to molecule resulting finally in a cellular response. These signal transduction cascades are highly regulated and often overlapping as evidenced by the existence of many protein kinases as well as phosphatases. It is currently believed that a number of disease states and/or disorders are a result of either aberrant activation or functional mutations in the molecular components of kinase cascades.

MAPKAP-K2 is a serine/threonine kinase that operates immediately downstream of the p38 within the stress-induced MAPK pathway (FIG. 1).

The p38 pathway is involved in transducing the effects of a variety of stress-related extracellular stimuli such as heat shock, UV light, bacterial lipopolysaccharide, and pro-inflammatory cytokines. Activation of this pathway results in the phosphorylation of transcription and initiation factors, and affects cell division, apoptosis, invasiveness of cultured cells and the inflammatory response (Martin-Blanco, Bioessays 22, 637-645 (2000)).

p38 itself activates a number of protein kinases other than the MAPKAP kinases such as Mnk1/2, PRAK and MSK1 (FIG. 1). The specific and/or overlapping functions of the majority of these downstream targets have yet to be resolved. This pathway has been of particular interest for the discovery of new anti-inflammatory agents. Previous strategies to intervene in this pathway have involved the development of selective inhibitors of p38 itself. Such inhibitors have proven efficacy in inhibiting pro-inflammatory cytokine production in cell-based models and demonstrated efficacy in animal models of chronic inflammatory conditions (Lee et al., Immunopharmacology 47, 185-201 (2000)). However, knockout of p38 expression in mouse models results in embryonic lethality, furthermore cells derived from such embryos have demonstrated a number of effects on fundamental cell responses. These observations indicate that caution should be applied to therapies involving the long-term dosing of humans with p38 inhibitors.

An alternative strategy for the development of anti-inflammatory agents could be the inhibition of this pathway at the level of MAPKAP-K2. Human MAPKAP-K2 has two proline-rich segments at its N-terminus followed by the kinase domain and the C-terminal regulatory domain. The kinase has low homology with other serine/threonine kinases except MAPKAP-K3 and 4. The C-terminal regulatory domain contains a bipartite nuclear localisation signal and a nuclear export signal. The crystal structure of inactive MAPKAP-K2 has been resolved (Meng, W. et al. J. Biol. Chem. 277, 37401-37405 (2002)). Activation of MAPKAP-K2 by p38 occurs via the selective phosphorylation of threonine residues 222 and 334 (Stokoe et al., EMBO J. 11, 3985-3994 (1992)). MAPKAP-K2 has an amphiphilic A-helix motif located within its C-terminal region that is likely to act to block the binding of substrates. The dual phosphorylation by p38 has been proposed to reposition this motif resulting in enhanced catalytic activity (You-Li et al., J. Biol. Chem. 270, 202-206 (1995)). MAPKAP-K2 is present in the nucleus of unstimulated cells and translocates to the cytoplasm upon cell activation. This kinase is known to phosphorylate a number of nuclear transcription factors as well as cytosolic proteins such as the heat shock proteins and 5-lipoxygenase (Stokoe et al., FEBS Lett. 313, 307-313 (1992), Werz, et al., Proc. Natl. Acad. Sci. USA 97, 5261-5266 (2000), Heidenreich, et al., J. Biol. Chem. 274, 14434-14443 (1999), Tan, et al., EMBO J. 15, 4629-4642 (1996), Neufeld, J. Biol. Chem. 275, 20239-20242 (2000)). All such substrates contain a unique amino acid motif (XX-Hyd-XRXXSXX, where Hyd is a bulky hydrophobic residue) that is required for efficient phosphorylation by MAPKAP-K2 (Stokoe et al., Biochem. J. 296, 843-849 (1993)).

Currently MAPKAP-K2 is the only p38 substrate for which a specific function has been identified. A specific role for MAPKAP-K2 in mediating the inflammatory response has been strongly indicated by the phenotype of the MAPKAP-K2-deficient mouse (MAPKAP-K2^{-/-}) (Kotlyarov, et al., Nature Cell Biol. 1, 94-97 (1999)). This mouse is viable and normal except for a significantly reduced inflammatory response. Recently it has also been shown that MAPKAP-K2 deficiency results in a marked neuroprotection from ischaemic brain injury (Wang et al., J. Biol. Chem. 277, 43968-43972 (2002)). MAPKAP-K2 is believed to regulate the translation and/or stability of important pro-inflammatory cytokine mRNAs. It is thought to perform this function via the phosphorylation of proteins that bind to the AU-rich elements found within untranslated regions of these cytokines. The identity of these proteins is currently under investigation.

MAPKAP-K2 therefore represents a targeted intervention point in the stress-induced kinase cascade for perturbation of the inflammatory response.

There exists a need for the provision of compounds that are inhibitors of MAPKAP-K2 kinases.

### DISCLOSURE OF THE INVENTION

As a result of much diligent research directed toward achieving the object stated above, the present inventors have completed the present invention upon discovering that the Pyrazolo[1,5-a]pyrimidine derivatives represented by formula (I) below and their pharmaceutically acceptable salts exhibit excellent kinase inhibiting activity.

In other words, the present invention provides as follows:
(1) A use of a compound of formula (I): 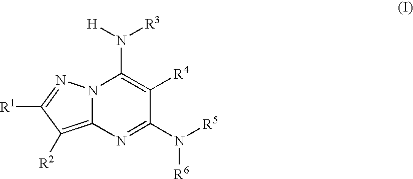
wherein R¹ is hydrogen
R² is hydrogen
R³ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl, optionally substituted heteroarylalkyl, optionally substituted arylalkenyl, optionally substituted heteroarylalkenyl, optionally substituted arylalkynyl, or optionally substituted heteroarylalkynyl;
R⁴ is hydrogen;
R⁵ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl, optionally substituted heteroarylalkyl, optionally substituted arylalkenyl, optionally substituted heteroarylalkenyl, optionally substituted arylalkynyl, or optionally substituted heteroarylalkynyl, optionally substituted heterocyclyl or optionally substituted heterocyclylalkyl;
R⁶ is hydrogen, C1-C8 optionally substituted alkyl, C2-C8 optionally substituted alkenyl, C2-C8 optionally substituted alkynyl or C3-C8 optionally substituted cycloalkyl;
or R⁵ and R⁶ together may be taken together with the nitrogen to which they are attached to form a mono or bicyclic heterocycle with 5-7 members in each ring and optionally containing, in addition to the nitrogen, one or two additional heteroatoms selected from N, O and S, the said mono or bicyclic heterocycle may optionally be substituted with one or more substituents;
or pharmaceutically acceptable salts, or other pharmaceutically acceptable biohydrolyzable derivatives thereof, including esters, amides, carbamates, carbonates, ureides, solvates, hydrates, affinity reagents or prodrugs thereof, in the manufacture of a medicament for use in inhibiting protein kinases.
(2) The use as (1), wherein R³ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl or optionally substituted heteroarylalkyl.
(3) The use as (2), wherein R³ is C2-C8 optionally substituted alkenyl, optionally substituted aryl or optionally substituted arylalkyl.
(4) The use as any one of (1) to (3), wherein R⁵ is C1-C8 optionally substituted alkyl, C2-C8 optionally substituted alkenyl, C2-C8 optionally substituted alkynyl, C3-C8 optionally substituted cycloalkyl, optionally substituted heterocyclyl or optionally substituted heterocyclylalkyl.
(5) The use as (4), wherein R⁵ is C3-C8 cycloalkyl substituted by NHR⁷, where R⁷ is optionally substituted heterocyclyl or optionally substituted heterocyclylalkyl.
(6) The use as any one of (1) to (5), wherein R⁶ is hydrogen or C1-C8 optionally substituted alkyl.
(7) The use as (6), wherein R⁶ is hydrogen.
(8) The use as any one of (1) to (7), wherein the medicament is for use as an inhibitor of MAPKAP-K2.
(9) The use as (8), wherein the medicament is for use in the prevention or treatment of a MAPKAP-K2-mediated disorder.
(10) The use as (9), wherein the MAPKAP-K2 mediated disorder is a neurological disorder (including dementia), an inflammatory disease, a disorder linked to apoptosis, particularly neuronal apoptosis, stroke, sepsis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation.
(11) The use as (10), wherein the disorder is a neurodegenerative disorder.
(12) The use as (11), wherein the neurodegenerative disorder is dementia, Alzheimer's disease, Parkinson's disease, Amyotrophic Lateral Sclerosis, Huntington's disease, senile chorea, Sydenham's chorea, hypoglycemia, head and spinal cord trauma including traumatic head injury, acute and chronic pain, epilepsy and seizures, olivopontocerebellar dementia, neuronal cell death, hypoxia-related neurodegeneration, acute hypoxia, glutamate toxicity including glutamate neurotoxicity, cerebral ischemia, dementia linked to meningitis and/or neurosis, cerebrovascular dementia, or dementia in an HIV-infected patient.
(13) The use as (10), wherein the disorder results from inflammation.
(14) The use as (13), wherein the disorder is inflammatory bowel disorder, bronchitis, asthma, acute pancreatitis, chronic pancreatitis, allergies of various types or Alzheimer's disease.
(15) The use as (10), wherein the disorder is an autoimmune disease.
(16) The use as (15), wherein the autoimmune disease is rheumatoid arthritis, systemic lupus erythematosus, glomerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.
(17) A method of treating or preventing a MAPKAP-K2-mediated disorder in an individual, which comprises administering to said individual at least one compound as defined in any one of (1) to (7) or a composition defined in (8) or (9).
(18) The method as (17), wherein the MAPKAP-K2 mediated disorder is a neurological disorder (including dementia), an inflammatory disease, a disorder linked to apoptosis, particularly neuronal apoptosis, stroke, sepsis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation.
(19) The method as (18), wherein the disorder is a neurodegenerative disorder.
(20) The method as (19), wherein the neurodegenerative disorder is dementia, Alzheimer's disease, Parkinson's disease, Amyotrophic Lateral Sclerosis, Huntington's disease, senile chorea, Sydenham's chorea, hypoglycemia, head and spinal cord trauma including traumatic head injury, acute and chronic pain, epilepsy and seizures, olivopontocerebellar dementia, neuronal cell death, hypoxia-related neurodegeneration, acute hypoxia, glutamate toxicity including glutamate neurotoxicity, cerebral ischemia, dementia linked to meningitis and/or neurosis, cerebrovascular dementia, or dementia in an HIV-infected patient.
(21) The method as (18), wherein the disorder results from inflammation.
(22) The method as (21), wherein the disorder is inflammatory bowel disorder, bronchitis, asthma, acute pancreatitis, chronic pancreatitis, allergies of various types or Alzheimer's disease.
(23) The method as (18), wherein the disorder is an autoimmune disease.
(24) The method as (23), wherein the autoimmune disease is rheumatoid arthritis, systemic lupus erythematosus, glomerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.
(25) The method as any one of (18) to (24), wherein one or more active agents is/are administered to the individual simultaneously, subsequently or sequentially to administering the compound.
(26) The method for determining the activity of the compounds as defined in any one of (1) to (7), comprising providing a system for assaying the activity and assaying the activity of a compound as defined in any of (1) to (7).
(27) The method as (26), wherein the assay is for the protein kinase inhibiting activity of the compound.
(28) A method of inhibiting the activity or function of a protein kinase, which comprises exposing a protein kinase to the compound as defined in any of (1) to (7).
(29) A method of inhibiting the activity or function of MAPKAP-K2, which comprises exposing MAPKAP-K2 to the compound as defined in any of (1) to (7).
(30) The method as (29) which is performed in a research model, in vitro, in silico, or in vivo such as in an animal model.

### BRIEF DESCRIPTION OF THE DRAWINGS

FIG. 1 shows the cascade of the p38 within the stress-induced MAPK pathway.

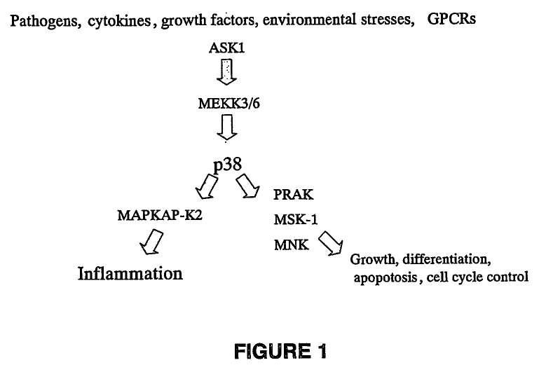

FIG. 2 shows a general reaction scheme for the preparation of compounds of Formula I. The invention will now be illustrated by the following non-limiting examples.

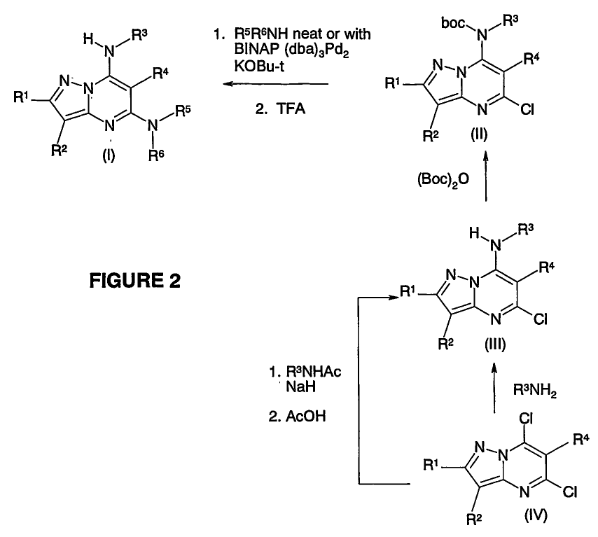

### BEST MODE FOR CARRYING OUT THE INVENTION

For the purposes of this invention, alkyl relates to both straight chain and branched alkyl radicals of 1 to 8 carbon atoms including but not limited to methyl, ethyl, n-propyl, isopropyl, n-butyl, sec-butyl, isobutyl, tert-butyl n-pentyl and n-hexyl. The term also encompasses cycloalkyl radicals of C3 to C8 carbon atoms including but not limited to cyclopropyl, cyclobutyl, CH₂-cyclopropyl, CH₂-cyclobutyl, cyclopentyl or cyclohexyl.

The term “alkenyl” means a straight chain or branched alkenyl radical of 2 to 8 carbon atoms and containing one or more carbon-carbon double bonds and includes but is not limited to ethylene, n-propyl-1-ene, n-propyl-2-ene, isopropylene, etc.

The term “alkynyl” means a straight chain or branched alkynyl radical of 2 to 8 carbon atoms and containing one or more carbon-carbon triple bonds and includes but is not limited to ethynyl, 2-methylethynyl etc.

“Aryl” means an aromatic 3-10 membered hydrocarbon containing one ring or being fused to one or more saturated or unsaturated rings including but not limited to phenyl, naphthyl, anthracenyl or phenanthracenyl.

“Heteroaryl” means an aromatic 3-10 membered aryl containing one or more heteroatoms selected from N, O or S and containing one ring or being fused to one or more saturated or unsaturated rings and;

“Heterocyclyl” means a 3-10 membered ring system containing one or more heteroatoms selected from N, O or S and includes heteroaryl. The heterocyclyl system can contain one ring or may be fused to one or more saturated or unsaturated rings; the heterocyclyl can be fully saturated, partially saturated or unsaturated and includes but is not limited to heteroaryl and heterocarbocyclyl. Examples of carbocyclyl or heterocyclyl groups include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, and trithiane.

Halogen means F, Cl, Br or I.

Suitable substituents include alkyl, cycloalkyl, heterocyclyl, alkenyl, alkynyl, alkoxy, aryloxy, halogen, hydroxy, NO₂, CN, CO₂R¹⁴, CONR¹⁴R¹⁵, NR¹⁴(CO)ₙR¹⁵, S(O)ₘR¹⁴; where R¹⁴ and R¹⁵, which may be the same or different, are hydrogen, alkyl or aryl; n is 0.1; m is 0 μl or 2.

Preferably, R³ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl or optionally substituted heteroarylalkyl;

More preferably R³ is C1-C8 optionally substituted alkenyl, optionally substituted aryl or optionally substituted arylalkyl

Preferably, R⁵ is C1-C8 optionally substituted alkyl, C2-C8 optionally substituted alkenyl, C2-C8 optionally substituted alkynyl, C3-C8 optionally substituted cycloalkyl, optionally substituted heterocyclyl or optionally substituted heterocyclylalkyl.

More preferably R⁵ is C3-C8 cycloalkyl substituted by NHR⁷, where R⁷ is optionally substituted heterocyclyl or optionally substituted heterocyclylalkyl.

Preferably R⁶ is hydrogen or C1-C8 optionally substituted alkyl. More preferably R⁶ is hydrogen.

As preferred combinations of the groups mentioned as preferred examples of R¹-R⁶ in formula I according to the invention, there may be mentioned the following combinations 1) or 2).
1) In formula I, wherein R¹ is hydrogen, R² is hydrogen, R³ is C6-C14 optionally substituted aryl, R⁴ is hydrogen, R⁵ is C3-C8 optionally substituted cycloalkyl and R⁶ is hydrogen.
2) In formula I, wherein R¹ is hydrogen, R² is hydrogen, R³ is C6-C14 optionally substituted aryl, R⁴ is hydrogen, R⁵ is optionally substituted heterocyclyl and R⁶ is hydrogen.

The compounds for use in the first aspect may be provided as a salt, preferably as a pharmaceutically acceptable salt of compounds of formula I. Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of ammonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases which are nontoxic and strong enough to form salts. Such organic bases are already well known in the art and may include amino acids such as arginine and lysine, mono-, di-, or trihydroxyalkylamines such as mono-, di-, and triethanolamine, choline, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl)aminomethane; and the like.

Salts may be prepared in a conventional manner using methods well known in the art. Acid addition salts of said basic compounds may be prepared by dissolving the free base compounds according to the first or second aspects of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The invention also extends to the use of a prodrug of the aforementioned compounds such as an ester or amide thereof. A prodrug is any compound that may be converted under physiological conditions or by solvolysis to any of the compounds of the invention or to a pharmaceutically acceptable salt of the compounds of the invention. A prodrug may be inactive when administered to a subject but is converted in vivo to an active compound of the invention.

The compounds for use according to the invention may contain one or more asymmetric carbon atoms and may exist in racemic and optically active forms. The compounds of the invention may exist in trans or cis form. The first aspect of the invention covers the use of all such compounds.

As specific examples of compounds of the formula I above there may be mentioned compounds listed in Table A below.

Wherein “Me” and “Ph” mean “methyl group” and “phenyl group” respectively.

TABLE A

| Com- | | | | | |
|---|---|---|---|---|---|
| pound | | | | | |
| No. | R¹ | R² | R⁴ | R³ | NR⁵R⁶ |
| 1 | H | H | H | 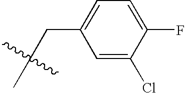 | 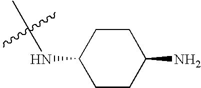 |
| 2 | H | H | H | 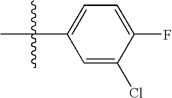 | 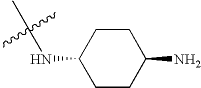 |
| 3 | H | H | H | 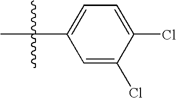 | 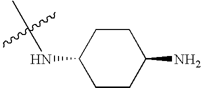 |
| 4 | H | H | H | 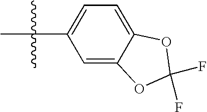 | 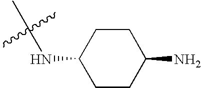 |
| 5 | H | H | H | 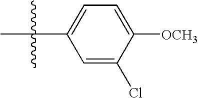 | 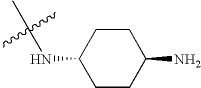 |
| 6 | H | H | H | 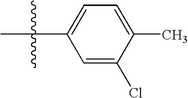 | 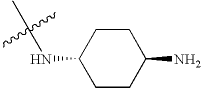 |
| 7 | H | H | H | 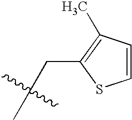 | 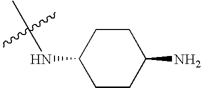 |
| 8 | H | H | H | 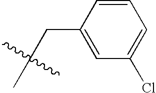 | 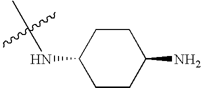 |
| 9 | H | H | H | 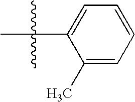 | 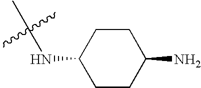 |
| 10 | H | H | H | 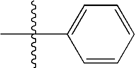 | 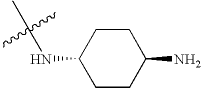 |
| 11 | H | H | H | 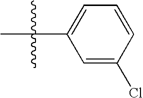 | 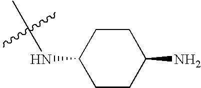 |
| 12 | H | H | H | 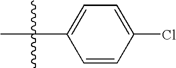 | 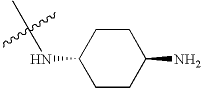 |
| 13 | H | H | H | 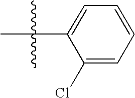 | 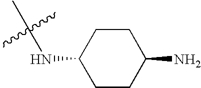 |
| 14 | H | H | H | 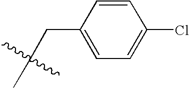 | 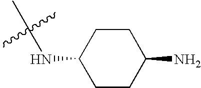 |
| 15 | H | H | H | 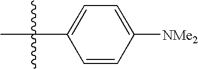 | 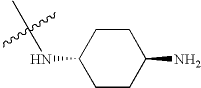 |
| 16 | H | H | H | 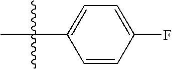 | 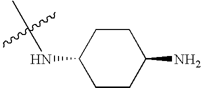 |
| 17 | H | H | H | 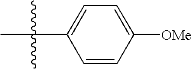 | 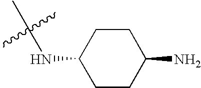 |
| 18 | H | H | H | 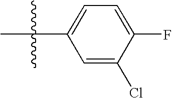 | 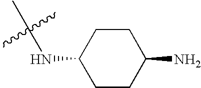 |
| 19 | H | H | H | 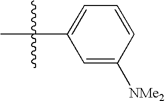 | 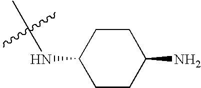 |
| 20 | H | H | H | 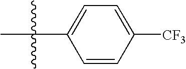 | 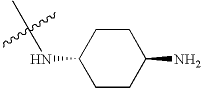 |
| 21 | H | H | H | 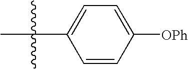 | 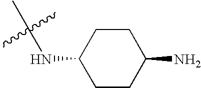 |
| 22 | H | H | H | 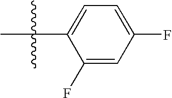 | 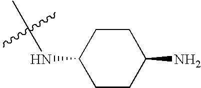 |
| 23 | H | H | H | 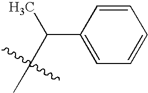 | 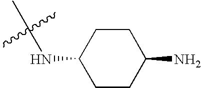 |
| 24 | H | H | H | 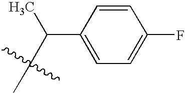 | 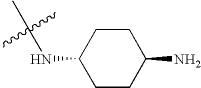 |
| 25 | H | H | H | 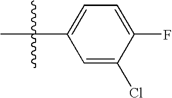 | 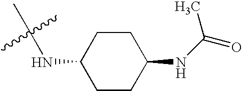 |
| 26 | H | H | H | 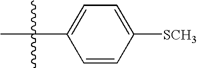 | 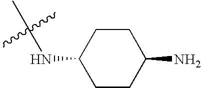 |
| 27 | H | H | H | 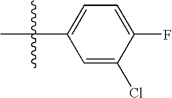 | 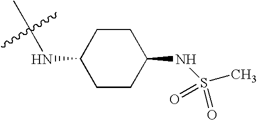 |
| 28 | H | H | H | 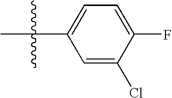 | 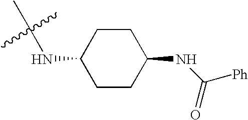 |
| 29 | H | H | H | 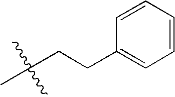 | 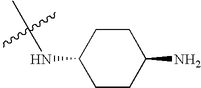 |
| 30 | H | H | H | 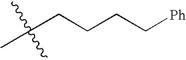 | 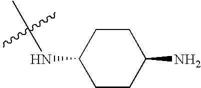 |
| 31 | H | H | H | 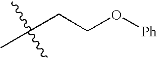 | 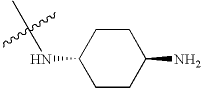 |
| 32 | H | H | H | 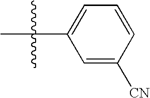 | 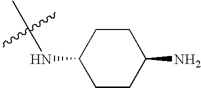 |
| 33 | H | H | H | 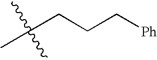 | 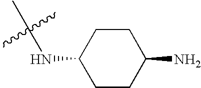 |
| 34 | H | H | H | 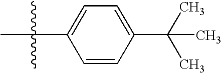 | 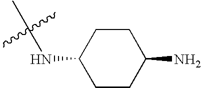 |
| 35 | H | H | H | 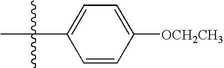 | 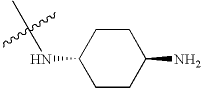 |
| 36 | H | H | H | 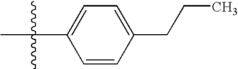 | 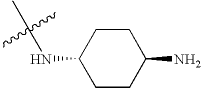 |
| 37 | H | H | H | 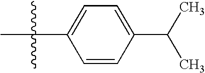 | 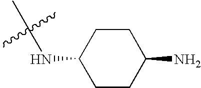 |
| 38 | H | H | H | 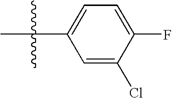 | 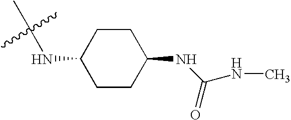 |
| 39 | H | H | H | 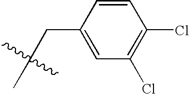 | 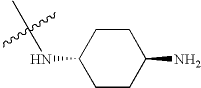 |
| 40 | H | H | H | 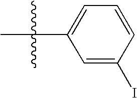 | 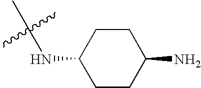 |
| 41 | H | H | H | 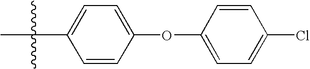 | 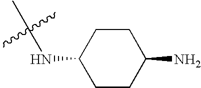 |
| 42 | H | H | H | 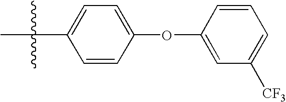 | 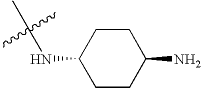 |
| 43 | H | H | H | 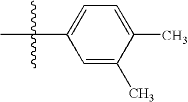 | 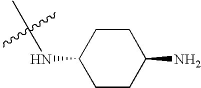 |
| 44 | H | H | H | 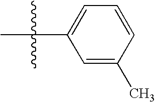 | 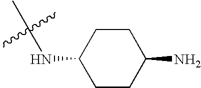 |
| 45 | H | H | H | 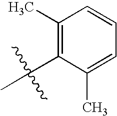 | 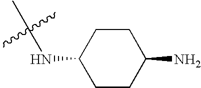 |
| 46 | H | H | H | 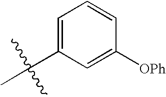 | 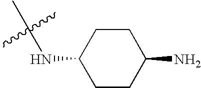 |
| 47 | H | H | H | 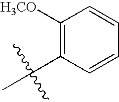 | 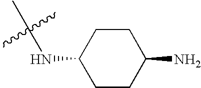 |
| 48 | H | H | H | 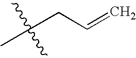 | 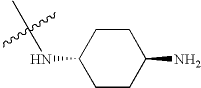 |
| 49 | H | H | H | 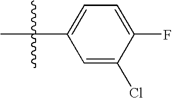 | 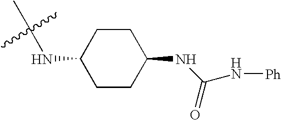 |
| 50 | H | H | H | 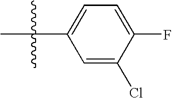 | 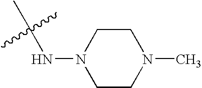 |
| 51 | H | H | H | 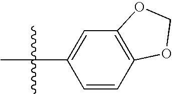 | 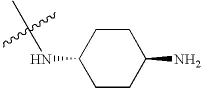 |
| 52 | H | H | H | 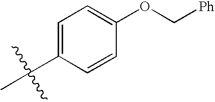 | 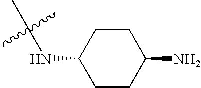 |
| 53 | H | H | H | 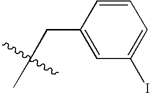 | 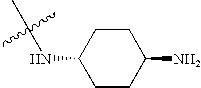 |
| 54 | H | H | H | 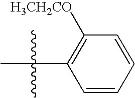 | 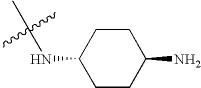 |
| 55 | H | H | H | 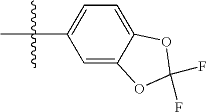 | 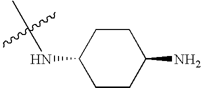 |
| 56 | H | H | H | 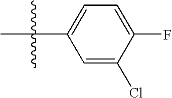 | 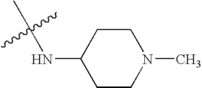 |
| 57 | H | H | H | 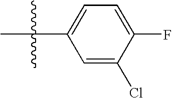 | 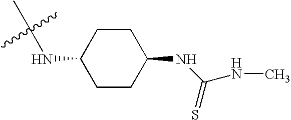 |
| 58 | H | H | H | 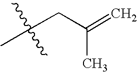 | 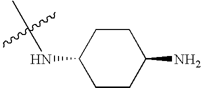 |
| 59 | H | H | H | 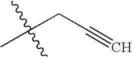 | 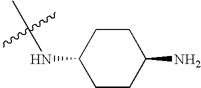 |
| 60 | H | H | H | 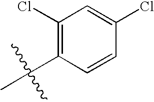 | 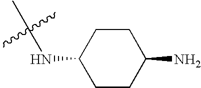 |
| 61 | H | H | H | 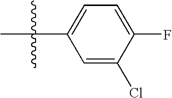 | 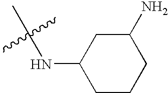 |
| 62 | H | H | H | 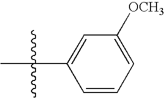 | 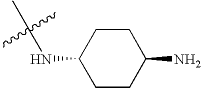 |
| 63 | H | H | H | 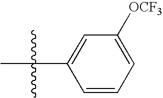 | 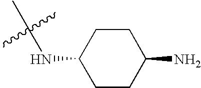 |
| 64 | H | H | H | 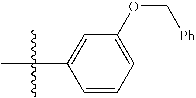 | 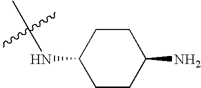 |
| 65 | H | H | H | 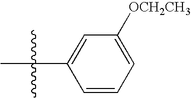 | 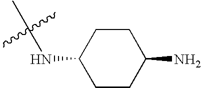 |
| 66 | H | H | H | 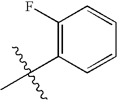 | 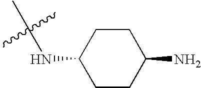 |
| 67 | H | H | H | 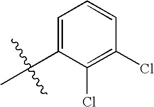 | 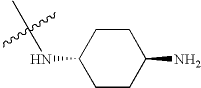 |
| 68 | H | H | H | 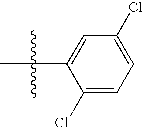 | 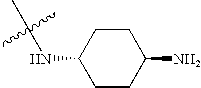 |
| 69 | H | H | H | 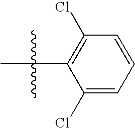 | 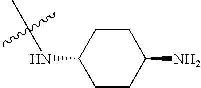 |
| 70 | H | H | H | 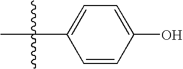 | 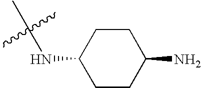 |
| 71 | H | H | H | 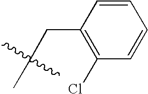 | 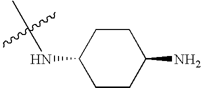 |
| 72 | H | H | H | 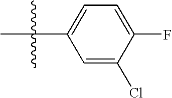 | 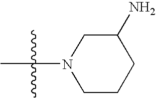 |
| 73 | H | H | H | 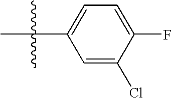 | 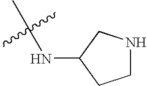 |
| 74 | H | H | H | 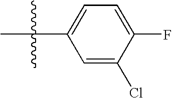 | 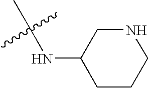 |
| 75 | H | H | H | 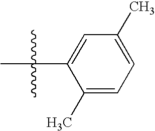 | 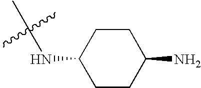 |
| 76 | H | H | H | 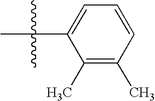 | 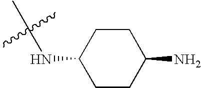 |
| 77 | H | H | H | 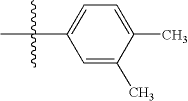 | 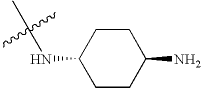 |
| 78 | H | H | H | 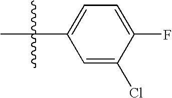 | 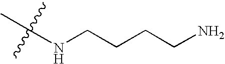 |
| 79 | H | H | H | 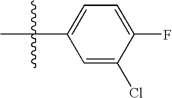 | 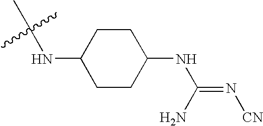 |
| 80 | H | H | H | 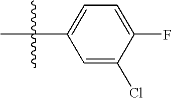 | 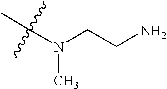 |
| 81 | H | H | H | 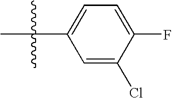 | 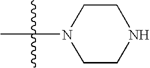 |
| 82 | H | H | H | 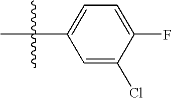 | 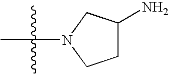 |
| 83 | H | H | H | 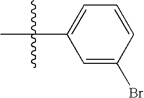 | 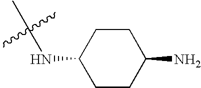 |
| 84 | H | H | H | 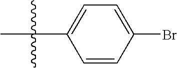 | 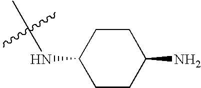 |
| 85 | H | H | H | 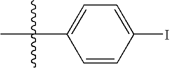 | 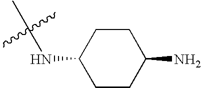 |
| 86 | H | H | H | 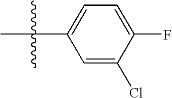 | 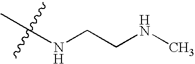 |
| 87 | H | H | H | 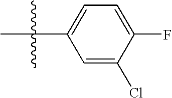 | 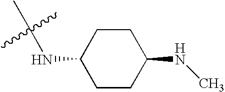 |
| 88 | H | H | H | 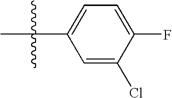 | 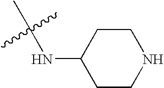 |
| 89 | H | H | H | 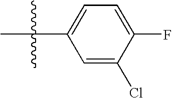 | 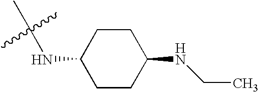 |
| 90 | H | H | H | 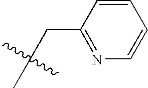 | 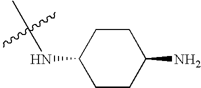 |
| 91 | H | H | H | 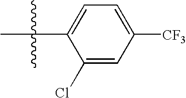 | 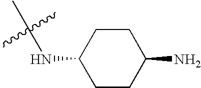 |
| 92 | H | H | H | 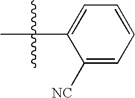 | 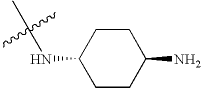 |
| 93 | H | H | H | 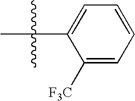 | 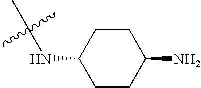 |
| 94 | H | H | H | 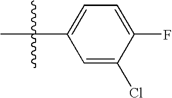 | 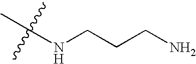 |
| 95 | H | H | H | 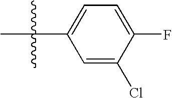 | 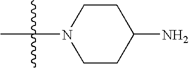 |
| 96 | H | H | H | 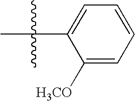 | 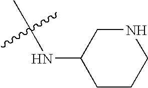 |
| 97 | H | H | H | 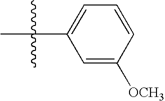 | 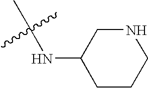 |
| 98 | H | H | H | 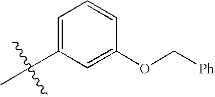 | 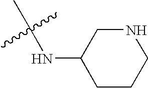 |
| 99 | H | H | H | 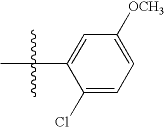 | 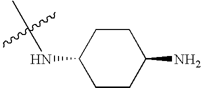 |
| 100 | H | H | H | 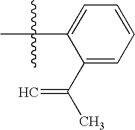 | 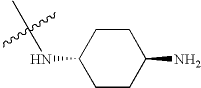 |
| 101 | H | H | H | 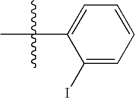 | 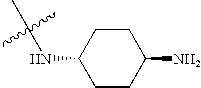 |
| 102 | H | H | H | 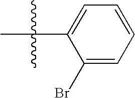 | 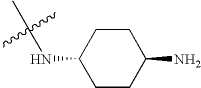 |
| 103 | H | H | H | 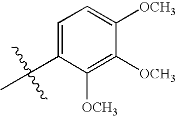 | 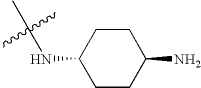 |
| 104 | H | H | H | 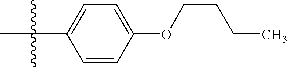 | 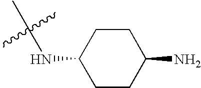 |
| 105 | H | H | H | 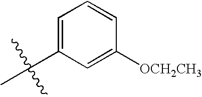 | 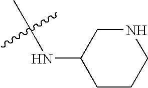 |
| 106 | H | H | H | 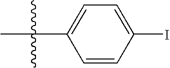 | 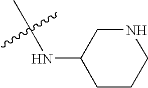 |
| 107 | H | H | H | 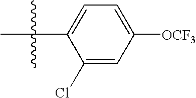 | 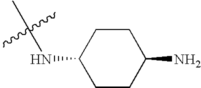 |
| 108 | H | H | H | 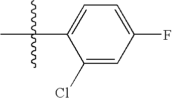 | 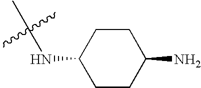 |
| 109 | H | H | H | 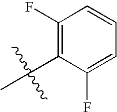 | 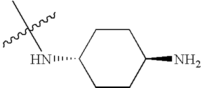 |
| 110 | H | H | H | 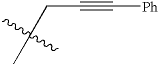 | 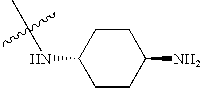 |
| 111 | H | H | H | 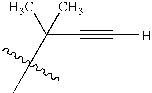 | 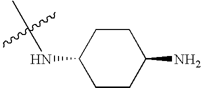 |
| 112 | H | H | H | 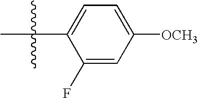 | 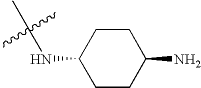 |
| 113 | H | H | H | 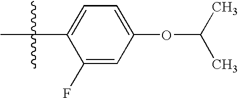 | 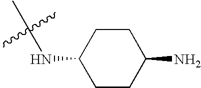 |
| 114 | H | H | H | 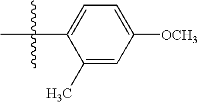 | 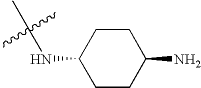 |
| 115 | H | H | H | 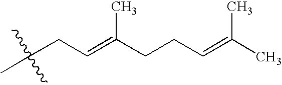 | 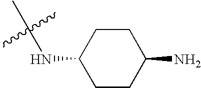 |
| 116 | H | H | H | 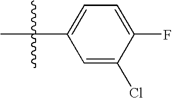 | 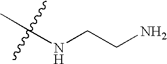 |
| 117 | H | H | H | 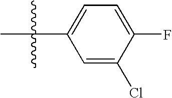 | 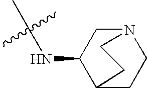 |
| 118 | H | H | H | 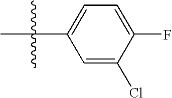 | 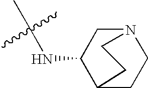 |
| 119 | H | H | H | 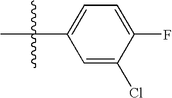 | 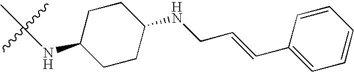 |
| 120 | H | H | H | 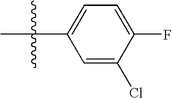 | 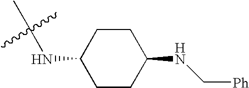 |
| 121 | H | H | H | 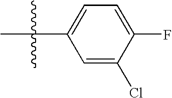 | 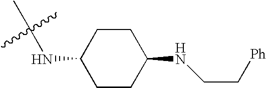 |
| 122 | H | H | H | 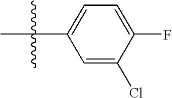 | 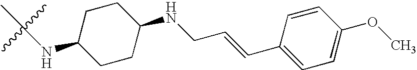 |
| 123 | H | H | H | 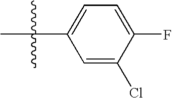 | 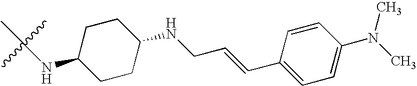 |
| 124 | H | H | H | 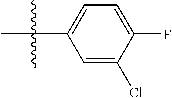 | 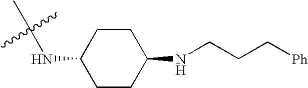 |
| 125 | H | H | H | 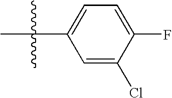 | 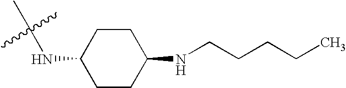 |
| 126 | H | H | H | 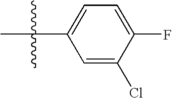 | 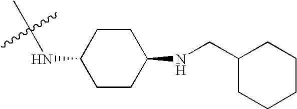 |
| 127 | H | H | H | 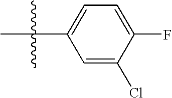 | 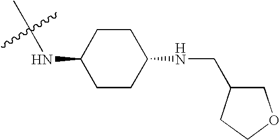 |
| 128 | H | H | H | 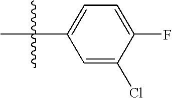 | 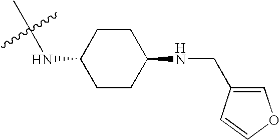 |
| 129 | H | H | H | 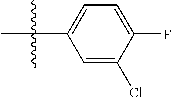 | 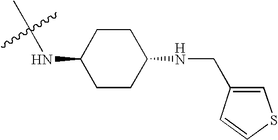 |
| 130 | H | H | H | 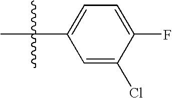 | 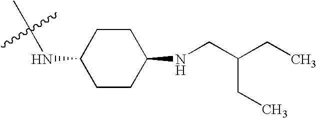 |
| 131 | H | H | H | 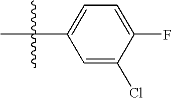 | 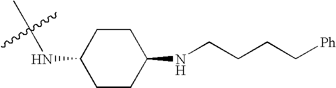 |
| 132 | H | H | H | 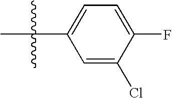 | 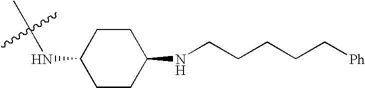 |
| 133 | H | H | H | 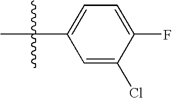 | 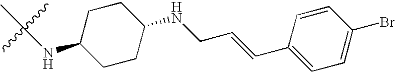 |
| 134 | H | H | H | 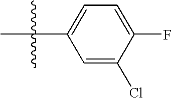 | 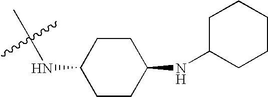 |
| 135 | H | H | H | 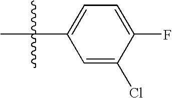 | 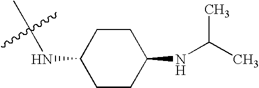 |
| 136 | H | H | H | 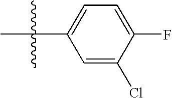 | 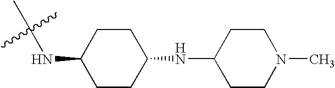 |
| 137 | H | H | H | 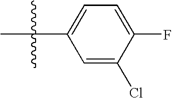 | 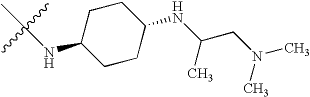 |
| 138 | H | H | H | 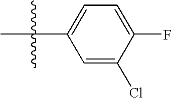 | 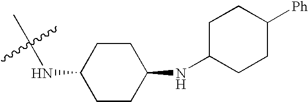 |
| 139 | H | H | H | 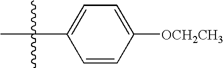 | 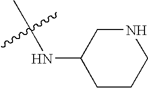 |
| 140 | H | H | H | 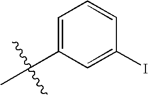 | 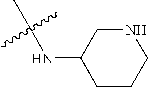 |
| 141 | H | H | H | 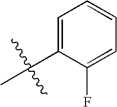 | 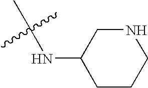 |
| 142 | H | H | H | 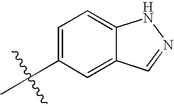 | 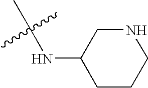 |
| 143 | H | H | H | 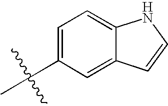 | 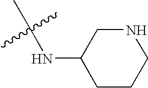 |
| 144 | H | H | H | 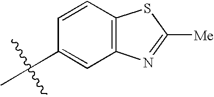 | 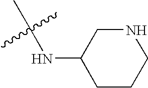 |
| 145 | H | H | H | 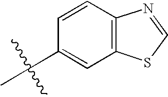 | 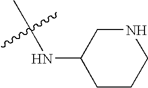 |
| 146 | H | H | H | 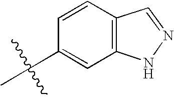 | 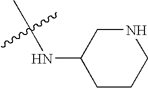 |
| 147 | H | H | H | 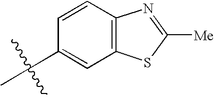 | 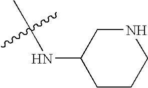 |
| 148 | H | H | H | 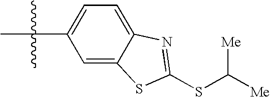 | 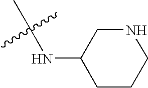 |
| 149 | H | H | H | 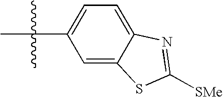 | 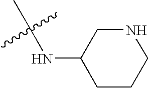 |
| 150 | H | H | H | 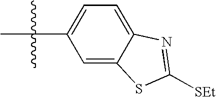 | 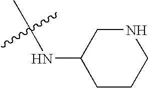 |
| 151 | H | H | H | 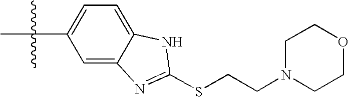 | 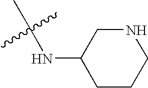 |
| 152 | H | H | H | 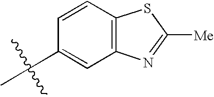 | 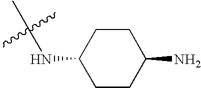 |
| 153 | H | H | H | 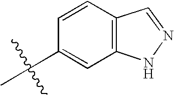 | 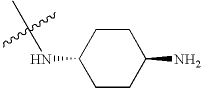 |
| 154 | H | H | H | 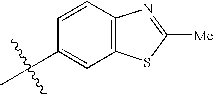 | 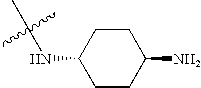 |
| 155 | H | H | H | 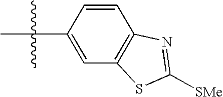 | 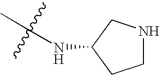 |
| 156 | H | H | H | 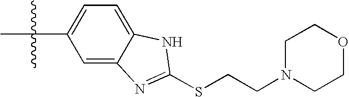 | 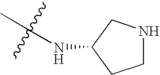 |
| 157 | H | H | H | 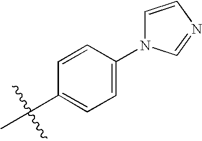 | 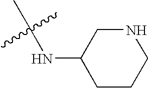 |
| 158 | H | H | H | 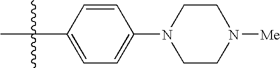 | 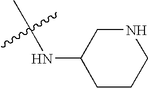 |
| 159 | H | H | H | 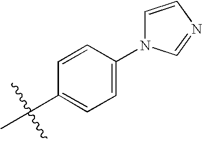 | 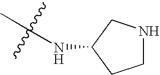 |
| 160 | H | H | H | 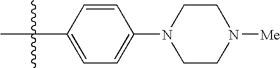 | 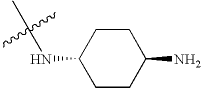 |
| 161 | H | H | H | 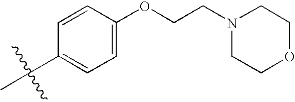 | 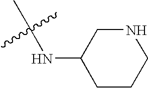 |
| 162 | H | H | H | 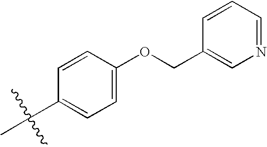 | 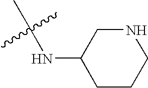 |
| 163 | H | H | H | 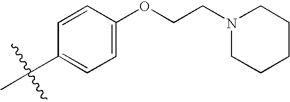 | 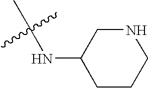 |
| 164 | H | H | H | 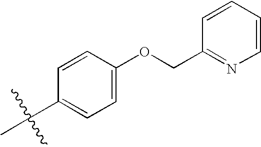 | 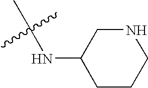 |
| 165 | H | H | H | 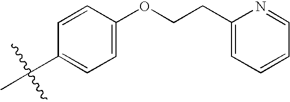 | 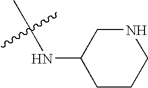 |
| 166 | H | H | H | 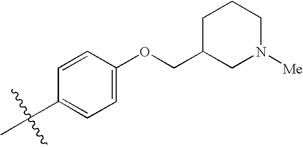 | 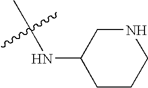 |
| 167 | H | H | H | 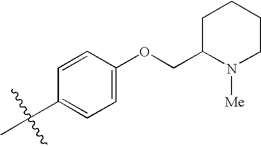 | 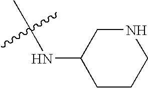 |
| 168 | H | H | H | 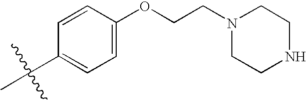 | 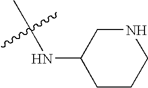 |
| 169 | H | H | H | 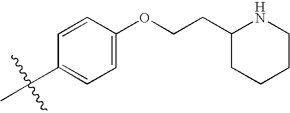 | 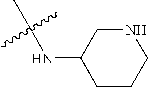 |
| 170 | H | H | H | 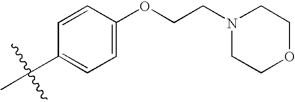 | 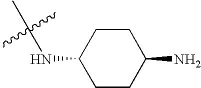 |
| 171 | H | H | H | 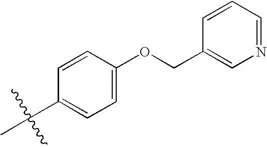 | 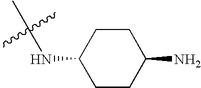 |
| 172 | H | H | H | 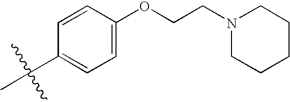 | 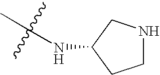 |
| 173 | H | H | H | 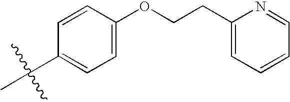 | 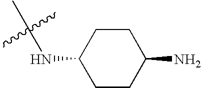 |
| 174 | H | H | H | 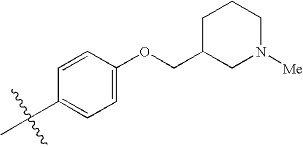 | 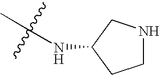 |
| 175 | H | H | H | 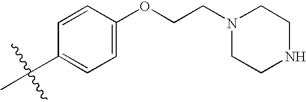 | 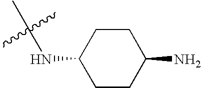 |
| 176 | H | H | H | 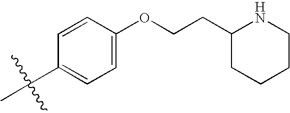 | 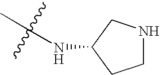 |
| 177 | H | H | H | 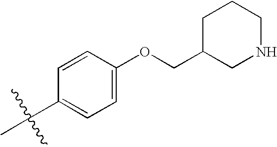 | 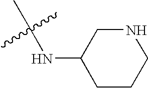 |
| 178 | H | H | H | 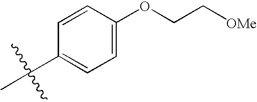 | 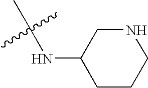 |
| 179 | H | H | H | 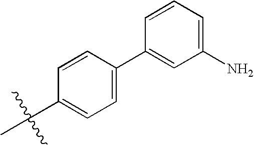 | 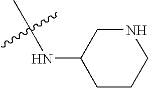 |
| 180 | H | H | H | 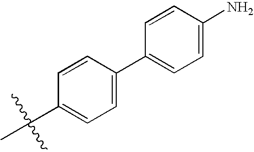 | 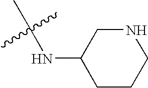 |
| 181 | H | H | H | 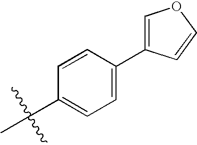 | 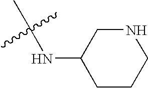 |
| 182 | H | H | H | 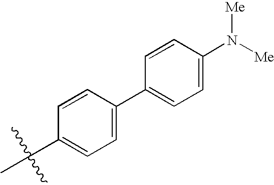 | 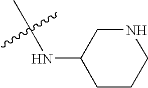 |
| 183 | H | H | H | 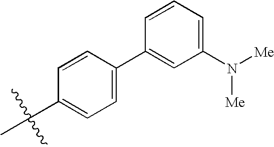 | 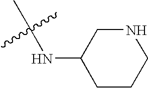 |
| 184 | H | H | H | 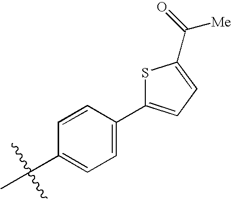 | 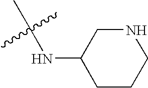 |
| 185 | H | H | H | 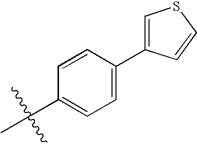 | 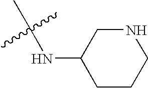 |
| 186 | H | H | H | 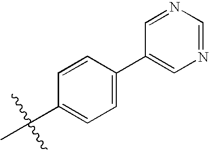 | 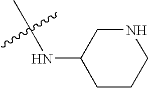 |
| 187 | H | H | H | 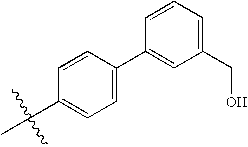 | 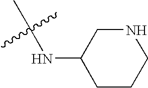 |
| 188 | H | H | H | 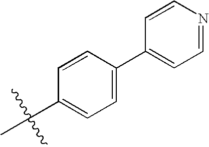 | 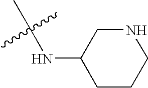 |
| 189 | H | H | H | 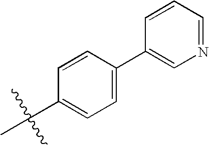 | 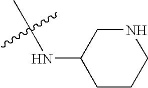 |
| 190 | H | H | H | 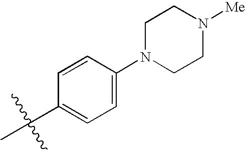 | 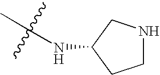 |
| 191 | H | H | H | 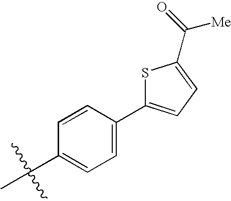 | 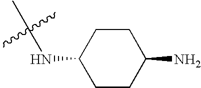 |
| 192 | H | H | H | 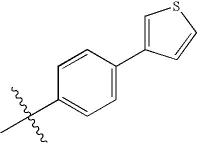 | 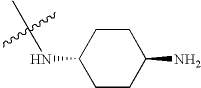 |
| 193 | H | H | H | 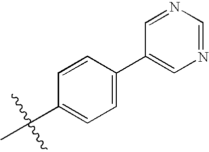 | 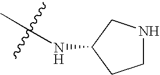 |
| 194 | H | H | H | 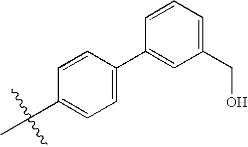 | 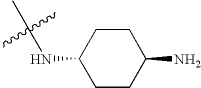 |
| 195 | H | H | H | 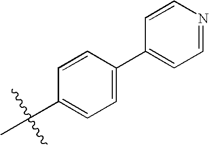 | 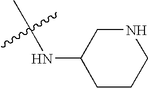 |
| 196 | H | H | H | 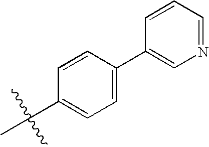 | 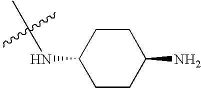 |
| 197 | H | H | H | 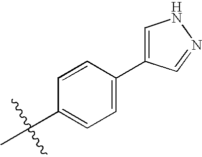 | 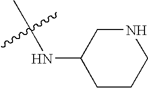 |
| 198 | H | H | H | 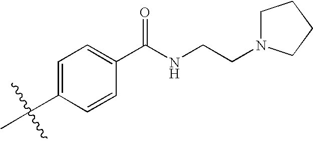 | 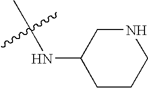 |
| 199 | H | H | H | 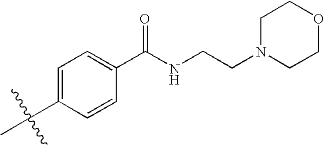 | 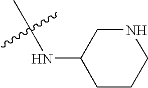 |
| 200 | H | H | H | 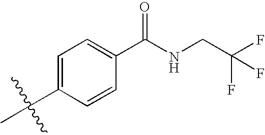 | 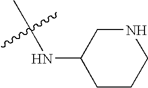 |
| 201 | H | H | H | 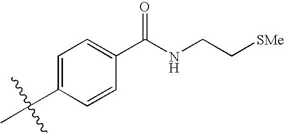 | 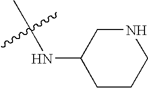 |
| 202 | H | H | H | 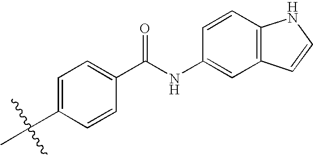 | 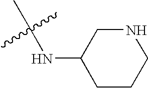 |
| 203 | H | H | H | 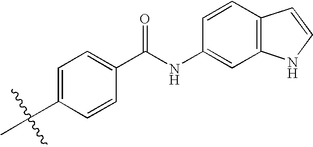 | 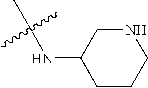 |
| 204 | H | H | H | 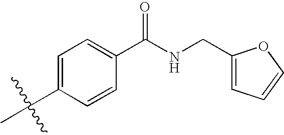 | 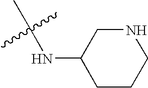 |
| 205 | H | H | H | 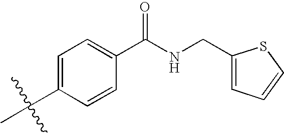 | 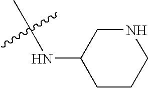 |
| 206 | H | H | H | 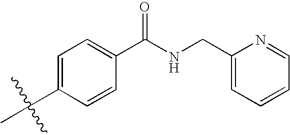 | 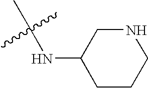 |
| 207 | H | H | H | 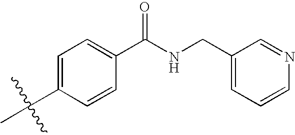 | 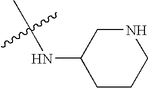 |
| 208 | H | H | H | 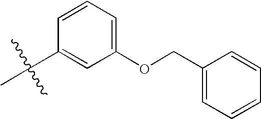 | 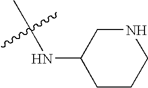 |
| 209 | H | H | H | 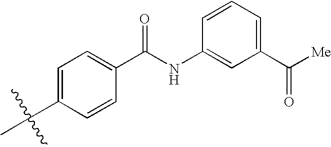 | 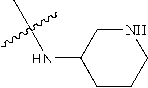 |
| 210 | H | H | H | 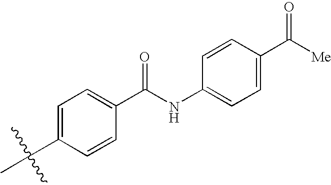 | 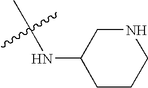 |
| 211 | H | H | H | 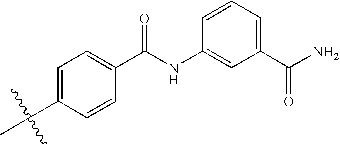 | 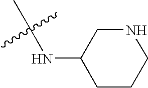 |
| 212 | H | H | H | 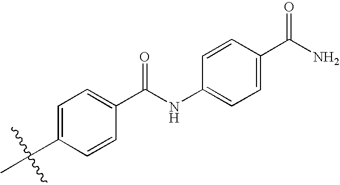 | 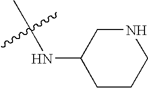 |
| 213 | H | H | H | 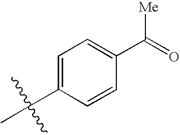 | 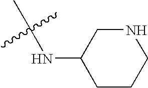 |
| 214 | H | H | H | 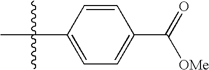 | 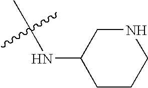 |
| 215 | H | H | H | 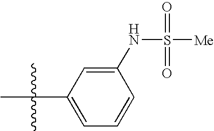 | 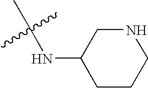 |
| 216 | H | H | H | 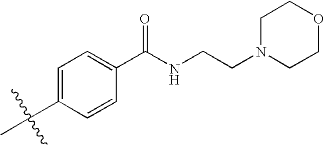 | 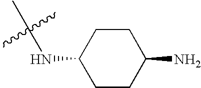 |
| 217 | H | H | H | 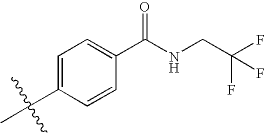 | 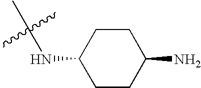 |
| 218 | H | H | H | 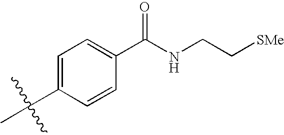 | 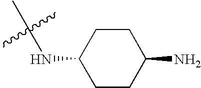 |
| 219 | H | H | H | 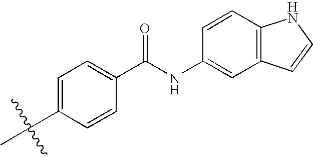 | 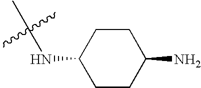 |
| 220 | H | H | H | 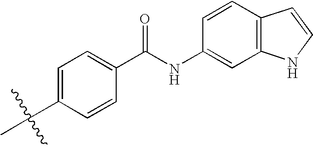 | 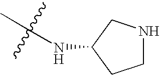 |
| 221 | H | H | H | 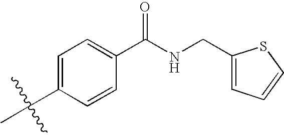 | 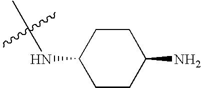 |
| 222 | H | H | H | 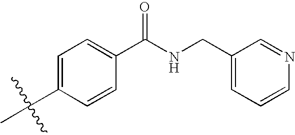 | 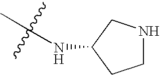 |
| 223 | H | H | H | 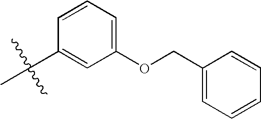 | 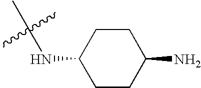 |
| 224 | H | H | H | 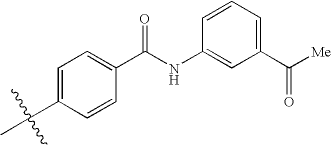 | 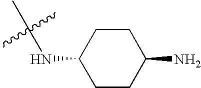 |
| 225 | H | H | H | 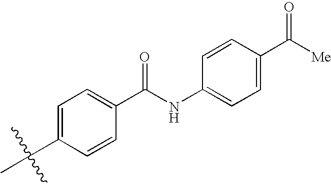 | 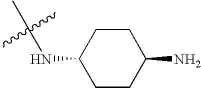 |
| 226 | H | H | H | 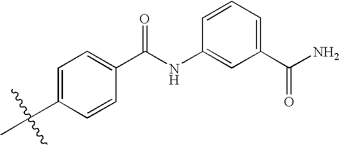 | 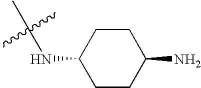 |
| 227 | H | H | H | 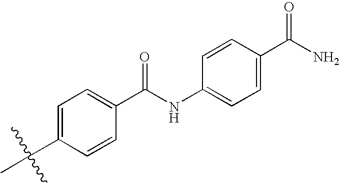 | 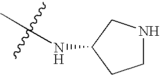 |
| 228 | H | H | H | 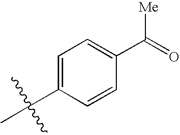 | 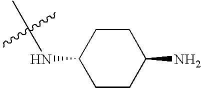 |
| 229 | H | H | H | 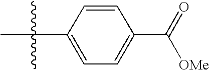 | 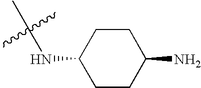 |

Suitably, the compounds as defined herein are inhibitors of MAPKAP-K2. For the purpose of this invention, an inhibitor is any compound which reduces or prevents the activity of the MAPKAP-K2 enzyme.

A “MAPKAP-K2-mediated disorder” is any disease or deleterious condition in which MAPKAP-K2 plays a role. Examples include neurological disorder (including dementia), inflammatory disease, a disorder linked to apoptosis, particularly neuronal apoptosis, stroke, sepsis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation.

The compounds as defined herein are particularly useful for the prevention or treatment of a neurodegenerative disorder. In particular, the neurodegenerative disorder results from apoptosis and/or inflammation. Examples of neurodegenerative disorders are: dementia; Alzheimer's disease; Parkinson's disease; Amyotrophic Lateral Sclerosis; Huntington's disease; senile chorea; Sydenham's chorea; hypoglycemia; head and spinal cord trauma including traumatic head injury; acute and chronic pain; epilepsy and seizures; olivopontocerebellar dementia; neuronal cell death; hypoxia-related neurodegeneration; acute hypoxia; glutamate toxicity including glutamate neurotoxicity; cerebral ischemia; dementia linked to meningitis and/or neurosis; cerebrovascular dementia; or dementia in an HIV-infected patient.

The compounds as defined herein can also be used to prevent or treat disorders resulting from inflammation. These include, for example, inflammatory bowel disorder, bronchitis, asthma, acute pancreatitis, chronic pancreatitis, allergies of various types, and possibly Alzheimer's disease. Autoimmune diseases which may also be treated or prevented by the compounds of the present invention include rheumatoid arthritis, systemic lupus erythematosus, glomerulonephritis, scleroderma, chronic thyroiditis, Graves's disease, autoimmune gastritis, diabetes, autoiminune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.

Compounds for use according to the present invention can be prepared as follows: by reaction of a compound of formula II, III, or VI as follows, wherein R¹-R⁶ are as defined above:
1) reacting a compound of the formula II 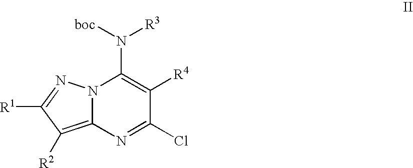
with a compound of the formula R⁵R⁶NH either in the absence or presence of metal catalysis under e.g. Buchwald conditions (J. Am. Chem. Soc. 116, 7901-7902 (1994)), and removal of the protecting group with for example CF₃CO₂H (for example as described in Protective Groups in Organic Synthesis, 3rd Ed, John Wiley & Sons Inc)
2) reacting a compound of the formula III 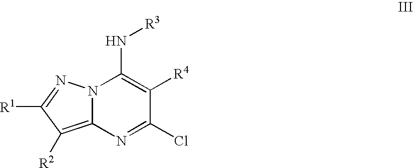
with a compound of the formula R⁵R⁶NH
3) reacting a compound of the formula III 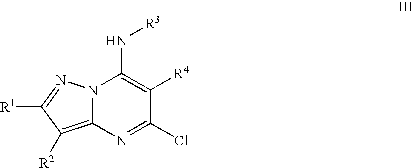
with a compound of the formula ((CH₃)₃COCO)₂O (for example as described in Protective Groups in Organic Synthesis, 3rd Ed, John Wiley & Sons Inc)
4) reacting a compound of the formula IV 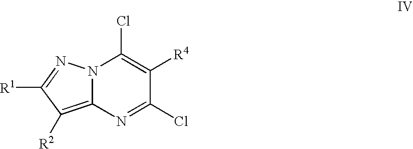
with a compound of the formula R³NH₂ or R³NHAc

A compound of formula I may undergo one or more further reactions to provide a different compound of formula I. For example, a compound may undergo a reduction, oxidation, elimination, substitution and/or addition reaction.

The compounds of formula IV are either known or can be prepared by methods analogous to those known for preparing analogous known compounds. Compounds of formula II and III include novel compounds and such novel compounds form an additional aspect of the invention.

Other methods will be apparent to the chemist skilled in the art, as will the methods for preparing starting materials and intermediates. The Examples also make apparent various methods of preparing compounds of the invention as well as starting materials and intermediates.

Medicaments as defined herein may also comprise one or more additional active agents, such as an anti-inflammatory agent (for example a p38 inhibitor, glutamate receptor antagonist, or a calcium channel antagonist), a chemotherapeutic agent and/or an antiproliferative agent.

Suitable carriers and/or diluents are well known in the art and include pharmaceutical grade starch, mannitol, lactose, magnesium stearate, sodium saccharin, talcum, cellulose, glucose, sucrose, (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

The medicaments may be administered by any convenient method, for example by oral (including by inhalation), parenteral, mucosal (e.g. buccal, sublingual, nasal), rectal or transdermal administration and the compositions adapted accordingly.

For oral administration, the composition can be formulated as liquids or solids, for example solutions, syrups, suspensions or emulsions, tablets, capsules and lozenges.

A liquid formulation will generally consist of a suspension or solution of the compound or physiologically acceptable salt in a suitable aqueous or non-aqueous liquid carrier(s) for example water, ethanol, glycerine, polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Compositions for oral administration may be designed to protect the active ingredient against degradation as it passes through the alimentary tract, for example by an outer coating of the formulation on a tablet or capsule.

Typical parenteral compositions consist of a solution or suspension of the compound or physiologically acceptable salt in a sterile aqueous or non-aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a pharmaceutically acceptable propellant. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal or vaginal administration are conveniently in the form of suppositories (containing a conventional suppository base such as cocoa butter), pessaries, vaginal tabs, foams or enemas.

Compositions suitable for transdermal administration include ointments, gels, patches and injections including powder injections.

Conveniently the composition is in unit dose form such as a tablet, capsule or ampoule.

Manufacture of the medicaments can be carried out by standard techniques well known in the art. The composition may be in any form including a tablet, a liquid, a capsule, and a powder or in the form of a food product, e.g. a functional food. In the latter case the food product itself may act as the pharmaceutically acceptable carrier.

A compound as defined herein may be administered simultaneously, subsequently or sequentially with one or more other active agent, such as an anti-inflammatory agent e.g. p38 inhibitor, glutamate receptor antagonist, calcium channel antagonist, a chemotherapeutic agent or an antiproliferative agent. For example, for acute treatment, a p38 inhibitor may be administered to a patient prior to administering a compound of the present invention.

The compounds as defined herein will normally be administered in a daily dosage regimen (for an adult patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a physiologically acceptable salt thereof calculated as the free base, the compound being administered 1 to 4 times per day. Suitably the compounds will be administered for a period of continuous therapy, for example for a week or more.

In a second aspect, the present invention provides a method of treating or preventing a MAPKAP-K2-mediated disorder in an individual, which comprises administering to said individual a compound as defined herein. The active compound is preferably administered in a cumulative effective amount. The individual may be in need of the treatment or prevention. Any of the MAPKAP-K2-mediated disorders discussed above may be the subject of treatment or prevention. One or more other active agents may be administered to the individual simultaneously, subsequently or sequentially to administering the compound. The other active agent may be an anti-inflammatory agent such as a p38 inhibitor, glutamate receptor antagonist, calcium channel antagonist, a chemotherapeutic agent or an antiproliferative agent.

In a third aspect, the present invention provides an assay for determining the activity of the compounds as defined herein, comprising providing a system for assaying the activity and assaying the activity of the compound. Preferably the assay is for the MAPKAP-K2 inhibiting activity of the compound. The compounds as defined herein may be assayed in vitro, in vivo, in silico, or in a primary cell culture or a cell line. In vitro assays include assays that determine inhibition of the kinase activity of activated MAPKAP-K2. Alternatively, in vitro assays may quantitate the ability of a compound to bind MAPKAP-K2 and may be measured either by radiolabelling the compound prior to binding, then isolating the inhibitor/MAPKAP-K2 complex and determining the amount of the radiolabel bound or by running a competition experiment where new inhibitors are incubated with MAPKAP-K2 bound to known radioligands. An example of an assay, which may be used, is Scintillation Proximity Assay (SPA), preferably using radiolabelled ATP. Another example is ELISA. Any type or isoform of MAPKAP-K2 may be used in these assays.

In an fourth aspect, the present invention provides a method of inhibiting the activity or function of a MAPKAP-12, which comprises exposing a MAPKAP-K2 to a compound or a composition of the first or fourth aspect of the present invention. The method may be performed in a research model, in vitro, in silico, or in vivo such as in an animal model. A suitable animal model may be a kainic acid model in rat or mice, traumatic brain injury model in rat, or MPTP in mice.

All features of each of the aspects apply to all other aspects mutatis mutandis.

### EXAMPLES

The invention will now be explained in greater detail by the following examples, with the understanding that the scope of the invention is not in any sense restricted by these examples.

### Example 1

[General Procedures for the Synthesis of Pyrazolo[1,5-a]pyrimidines of General Formula (III)]

a) To a solution of 5,7-dichloropyrazolo[1,5-a]pyrimidine (IV) (2 g) and triethylamine (2 equivalents) in 2-propanol (20 ml) was added the amine R³NH₂ (1 or 1.1 equivalents) and the mixture was stirred at room temperature overnight. The mixture was concentrated in vacuo and the residue was then partitioned between water and dichloromethane. The organic phase was washed twice with water and the combined aqueous phases back-extracted with dichloromethane. The combined organic layers were combined, washed with brine and dried over Na₂SO₄. Removal of the solvent in vacuo yielded the precursor (III). (Purification performed—normally the products did not require any further purification, if they did, they were recrystallised. Analysis performed—NMR, HPLC and MS.)

Should the above room-temperature reaction not occur satisfactorily, the following may be applied:

b) To a solution of 5,7-dichloropyrazolo[1,5-a]pyrimidine (IV) (2 g) in 2-propanol (25 ml) containing N,N-diisopropylethylamine (2 equivalents) was added the amine R³NH₂ (1.2 equivalents). The reaction was heated overnight at 80° C. and the solvent removed in vacuo. The residue was partitioned between water and dichloromethane and the organic phase was washed with water, brine and dried over MgSO₄. Removal of the solvent in vacuo yielded the product.

In those cases where R³NH₂ is a hindered or weakly nucleophilic aniline the following procedure may be applied:

c) To a solution of 2-methylacetanilide (2.2 mmol) in toluene (3 ml) at room temperature was added sodium hydride (3 mmol) after the addition the mixture was heated until effervescence ceased and the solution became homogenous. 5,7-Dichloropyrazolo[1,5-a]pyrimidine (IV) (1 mmol) was added and the mixture heated at reflux for 5 h. (The solution becomes heterogeneous during this time). Upon cooling, acetic acid (1 ml) and water (1 ml) were cautiously added and the mixture was stirred for 15 min. The solvent was removed in vacuo and the residual acetic acid removed by azeotropic evaporation with toluene, (3×). The residue was partitioned between water and ethyl acetate. The organic phase was washed (water and brine) and dried. The solvent was removed in vacuo and the residue was chromatographed to afford the desired compound (III). Typical unoptimised yields for c) 50-70%. The Rf of starting material (IV) and product (III) are chromatographically indistinguishable, making complete reaction difficult to determine. It appears that at least 5 h is required for significant reaction to occur.

| Compound | | | | | |
|---|---|---|---|---|---|
| No | R¹ | R² | R⁴ | R³ | Mp (° C.) |
| IIIA | H | H | H | 2-Me | 119-121 |
| | | | | phenyl | |
| IIIB | H | H | H | 2,4-Cl₂ | 120-128 |
| | | | | phenyl | |

### Example 2

[General Procedure for the Synthesis of Pyrazolo[1,5-a]pyrimidines of General Formula (II)]

To a solution of the precursor (II) formed above (2 g) in 1,4-dioxane (10 ml) was added di-tert-butyl dicarbonate (2 equivalents) in 1,4-dioxane (10 ml) followed by 4-dimethylaminopyridine (cat). The reaction was stirred at room temperature overnight and if starting material was detected by TLC, the reaction was left for longer. The mixture was concentrated in vacuo and the residue was then partitioned between water and dichloromethane. The organic phase washed with 10% citric acid, water and brine and then dried over MgSO₄. Removal of the solvent it vacuo gave the Boc protected intermediate (II). (Purification performed—filter column to remove any residual 4-dimethylaminopyridine. Analysis performed—NMR, HPLC and MS.)

| Compound | | | | | |
|---|---|---|---|---|---|
| No | R¹ | R² | R⁴ | R³ | ¹H NMR(CDCl₃) |
| IIA | H | H | H | 2-Me | 1.38(9H, s, tBu), |
| | | | | phenyl | 2.3(3H, s, CH₃), |
| | | | | | 6.4(1H, s, Het-H), |
| | | | | | 6.44(1H, s, Het-H), |
| | | | | | 7.15-7.34(4H, |
| | | | | | m, ArH), 8.15(1H, s, |
| | | | | | Het-H) |
| IIC | H | H | H | 2-F phenyl | 1.4(9H, s, tBu), |
| | | | | | 6.67(1H, m, 2Het-H), |
| | | | | | 7.08-7.4(4H, m, |
| | | | | | ArH) 8.17(1H, s, |
| | | | | | Het-H) |

### Example 3

[General Procedures for the Synthesis of Pyrazolo[1,5-a]pyrimidines of General Formula (I)]

a) An intimate mixture of the Boc protected intermediate (II) (100 mg) and trans-1, 4-cyclohexanediamine (1.5 g) were heated together at 80-85° C. for 90 min, then cooled. The crude material was then partitioned between dichloromethane and saturated NaHCO₃ solution. The organic phase is then separated and washed with water. Dried over MgSO⁴ and concentrated in vacuo. The crude material dissolved in dichloromethane (10 ml) and trifluoroacetic acid (5 ml). Stirred for 1 h at room temperature, then evaporated in vacuo. The residue was partitioned between saturated NaHCO₃ and dichloromethane, the organic phase was separated, dried over MgSO₄ then subjected to column chromatography over silica gel. Eluent dichloromethane, then gradient elution up to 95% dichloromethane+5% (10 M NH₃ in methanol). Typical purified yield 20 mg

b) An intimate mixture of the Boc protected intermediate (II) (100 mg) and trans-1, 4-cyclohexanediamine (1.5 g) were heated together at 80-85° C. for 18 hr then cooled. The crude material was then partitioned between dichloromethane and saturated NaHCO₃ solution. The organic phase is then separated and washed with water. Dried over MgSO₄ and concentrated in vacuo. The crude product subjected to column chromatography over silica gel. Eluent dichloromethane, then gradient elution up to 95% dichloromethane+5% (10 M NH₃ in methanol). Typical purified yield 20 mg.

c) The intermediate (II) (0.1 mmol) was dissolved in toluene (1 ml) and the amine (1.2 equivalents) was added. Tris(dibenzylideneacetone)dipalladium (0) (2 mol %), 2,2′-bis(diphenylphosphino)-1,1′-binaphthyl (4 mol %) and sodium tert-butoxide (1.2 equivalents) were added sequentially under an atmosphere of nitrogen. The reaction was heated and agitated overnight at 80° C. following which the reaction was filtered through a 0.45 micron filter. The solvent was removed in vacuo and the residue was resuspended in dichloromethane (2 ml). Trifluoroacetic acid (0.8 ml) was added and the reactions allowed to stand for 1 h at room temperature. The mixture was evaporated to dryness, in vacuo, and the resultant residue was dissolved in N,N-dimethylformamide (1 ml), filtered and purified by prep-HPLC to give the product (I). (Analysis performed—LC/MS.)

d) Further elaborations of compounds of General Formula (I)

i) Acylations with acid halides, sulfonyl halides, isocyanates and isothicyanates

To a solution of Compound 2 (50 mgs) in dichloromethane (10 ml) was added triethylamine (1.1 equivalents) followed by the dropwise addition of the acid halide, sulfonyl halide, isocyanate or isothicyanate (1.05 equivalents). The mixture was stirred for 1-2 hours the washed with water, dried over MgSO₄, the solvent was removed in vacuo then the residue subjected to column chromatography over silica gel. Eluent dichloromethane, then gradient elution up to 95% dichloromethane+5% (10 M NH₃ in methanol) to afford compound, for example

| | | Mp (° C.)/or for |
|---|---|---|
| Compound No | NR⁵R⁶ | gums M⁺, M⁻ |
| 25 | trans-4-Acetylamino-c- | 239-241(d) |
| | hexylamine | |
| 27 | trans-4-Methylsulphonylamino-c- | Gum, 453, 451 |
| | hexylamine | |
| 38 | trans-4-MeNHCONH-c- | 233-238 |
| | hexylamine | |
| 57 | trans-4-MeNHCSNH-c- | 167-169 |
| | hexylamine | |

ii) Reductive Aminations

To a solution of Compound 2 (50 mgs) in tetrahydrofuran (5 ml) was added cyclohexanone (1.1 equivalents) and the reaction was heated overnight at 60° C. To the cooled mixture was then added sodium cyanoborohydride (5 equivalents) and stirred at ambient temperature for 2 hours. The mixture was evaporated to dryness, in vacuo, and the resultant residue dissolved in water and ethyl acetate. The organic phase was separated, dried over MgSO₄ then subjected to column chromatography over silica gel. Eluent dichloromethane, then gradient elution up to 95% dichloromethane+5% (10 M NH₃ in methanol) to afford compound 134, mp 85-87° C., 20 mg

Compounds of general formula (I) prepared by the above procedures are recorded in Table B. The numbers assigned to each of the compounds in Table B correspond to the Compound Nos. of the compounds listed as specific examples in Table A above. Compounds were characterised by mass spectrometry using single quadrupole instrumentation with an electrospray source. M+H indicates values obtained for compound molecular mass (M) with proton (H) capture and M-H compound molecular mass (M) with proton (H) loss. Melting points (mp) are uncorrected; (d) denotes decomposition at or near the melting point. Compounds which were not solids were gums.

TABLE B

ESI/MS

| Compound No. | Mp (° C.) | M + H | M − H |
|---|---|---|---|
| 1 | 171-173 | 389 | |
| 2 | 144-146(d) | 375 | 373 |
| 3 | gum | 391 | |
| 4 | 140-143(d) | 403 | |
| 5 | 138-141(d) | 387 | |
| 6 | gum | 371 | |
| 7 | gum | 357 | |
| 8 | 152-155(d) | | |
| 9 | 175-177 | 337 | |
| 10 | 88-89(d) | 323 | |
| 11 | 89-92(d) | 357 | |
| 12 | 158-161(d) | 357 | 355 |
| 13 | 153-156(d) | 357 | |
| 14 | gum | 371 | |
| 15 | gum | 366 | |
| 16 | gum | 341 | 339 |
| 17 | gum | 353 | 351 |
| 18 | gum | 375 | 373 |
| 19 | gum | 363 | 364 |
| 20 | 141-144(d) | 391 | 389 |
| 21 | gum | 415 | 413 |
| 22 | gum | 359 | 357 |
| 23 | 96-98(d) | | |
| 24 | 97-102(d) | | |
| 25 | 239-241(d) | 417 | 415 |
| 26 | 201-201(d) | 368 | |
| 27 | gum | 453 | 451 |
| 28 | 211-214(d) | 479 | 477 |
| 29 | 131-134 | 351 | |
| 30 | Gum | 379 | 377 |
| 31 | 135-138 | 367 | |
| 32 | Gum | 348 | 346 |
| 33 | Gum | 365 | |
| 34 | Gum | 379 | |
| 35 | 183-186 | 367 | |
| 36 | 181-183 | 365 | 363 |
| 37 | 87-92(d) | 365 | 363 |
| 38 | 233-238 | 431 | 430 |
| 39 | Gum | 407 | |
| 40 | 114-118(d) | 449 | 447 |
| 41 | 85-90(d) | 449 | |
| 42 | Gum | 483 | 481 |
| 43 | 87-92(d) | 351 | |
| 44 | 68-72(d) | 337 | |
| 45 | 251-254(d) | 351 | |
| 46 | 77-81(d) | 415 | |
| 47 | 78-82(d) | 353 | |
| 48 | Gum | 287 | |
| 49 | 227-228 | 494 | |
| 50 | Gum | 376 | |
| 51 | Gum | 367 | 365 |
| 52 | 158-162(d) | 429 | 427 |
| 53 | 192-194 | 461 | 463 |
| 54 | 92-96(d) | 367 | 365 |
| 55 | 94-98(d) | 403 | |
| 56 | 89-91 | 375 | |
| 57 | 167-169 | 448 | 446 |
| 58 | Gum | 301 | 299 |
| 59 | Gum | 285 | |
| 60 | Gum | 391 | 389 |
| 61 | gum | 375 | 373 |
| 62 | 64-66 | 353 | 351 |
| 63 | 62-65 | 407 | 405 |
| 64 | 80-83 | 429 | 427 |
| 65 | 86-88 | | |
| 66 | 129-130 | | |
| 67 | 163-167 | | |
| 68 | 95-100 | | |
| 69 | 217-219 | | |
| 70 | 184-188 | 339 | 337 |
| 71 | gum | 371 | |
| 72 | 175-177 | | |
| 73 | gum | 347 | |
| 74 | gum | 361 | |
| 75 | 175-177 | | |
| 76 | 95-100 | | |
| 77 | 85-90 | | |
| 78 | gum | 349 | |
| 79 | 80(d) | | |
| 80 | 149-150 | 335 | |
| 81 | 230-232 | 347 | |
| 82 | 218-219 | 347 | |
| 83 | 90-100 | | |
| 84 | 164-166 | | |
| 85 | 166-168 | | |
| 86 | gum | 335 | |
| 87 | gum | 389 | |
| 88 | 105-106 | 361 | 359 |
| 89 | 172-173 | 403 | 401 |
| 90 | Gum | 338 | |
| 91 | 100-105 | 425 | 423 |
| 92 | 130-140 | 348 | 346 |
| 93 | 100-105 | 391 | 389 |
| 94 | Gum | 335 | 333 |
| 95 | 155-157 | 361 | 359 |
| 96 | 55-57 | 339 | 337 |
| 97 | 60-63 | 339 | 337 |
| 98 | 60-62 | 415 | 413 |
| 99 | Gum | 387 | 385 |
| 100 | 66-71 | 363 | 361 |
| 101 | 88-91 | 449 | 447 |
| 102 | 120-123 | 401/403 | 399/401 |
| 103 | 216-219 | 413 | 411 |
| 104 | 155-157 | 395 | 393 |
| 105 | Gum | 353 | 351 |
| 106 | 95-97 | 435 | 433 |
| 107 | Gum | 441 | |
| 108 | 106-110 | 375 | |
| 109 | 98-106 | 359 | 357 |
| 110 | 103-106 | 361 | 359 |
| 111 | Gum | 313 | |
| 112 | 119-121 | 371 | 369 |
| 113 | 150-153 | 399 | 397 |
| 114 | 178-180 | 367 | |
| 115 | 80-82 | 383 | 381 |
| 116 | Gum | 321 | 319 |
| 117 | 69-71 | 387 | 385 |
| 118 | 120-130 | 387 | 385 |
| 119 | 52-54 | 491 | 489 |
| 120 | Gum | 465 | 463 |
| 121 | Gum | 479 | |
| 122 | Gum | 521 | 519 |
| 123 | 116-120 | | 532 |
| 124 | 58-61 | 493 | 491 |
| 125 | 207-210 | 445 | 443 |
| 126 | 65-69 | 471 | 469 |
| 127 | Gum | 459 | 457 |
| 128 | 48-51 | 455 | 453 |
| 129 | 60-70 | 471 | 469 |
| 130 | Gum | 459 | 457 |
| 131 | Gum | | 505 |
| 132 | Gum | | 519 |
| 133 | 72-74 | | 596 |
| 134 | 85-87 | | 455 |
| 135 | 133-135 | | 415 |
| 136 | Gum | | 470 |
| 137 | 55-60 | | 458 |
| 138 | Gum | | 531 |
| 139 | 121-124 | 353 | 351 |
| 140 | 130-134 | 435 | 433 |
| 141 | 202-204 | 327 | 325 |

MAPKAP-Kinase 2 Assay
[Compound Preparation]

Compounds are dissolved in DMSO at a concentration of 3 mM and stored in aliquots at −20° C. Compounds in DMSO from these stock aliquots are diluted in 30% DMSO to produce initial working stock solutions of 1 mM and 3 mM. Both of these stock solutions are then subjected to 1:10 serial dilutions in 30% DMSO in order to prepare 3000, 1000, 300, 100, 30, 10, 3, 1, 0.1, 0.01 μM stock solutions. 5 μl of each stock solution is used per 50 μl reaction to give final assay concentrations of 300, 100, 30, 10, 3, 1, 0.3, 0.1, 0.01, 0.001 μM.

[Assay]

The kinase reaction is conducted in a round-bottomed polypropylene 96-well plate. MAPKAP-K2 is diluted to 25 mU/μl in diluent buffer (50 mM Tris/HCl. pH7.5, 0.1 mM EGTA, 0.1% (v/v) β-mercaptoethanol, 1 mg/ml BSA). 5 μl compound or 30% DMSO is added to each well followed by 25 μl substrate cocktail (10 μM ATP, 30 μM peptide (KKLNRTLSVA), 0.5 μCi ³³P-γ-ATP in 50 mM Tris pH7.5, 0.1 mM EGTA, 10 mM Mg-acetate, 0.1% BME). The reaction is initiated with the addition of 20 μl enzyme solution per well or 20 μl diluent buffer without enzyme. The plate is shaken for 10 sec and then left at room temperature for 30 min. The reaction is terminated with 50 μl 150 mM phosphoric acid. 90 μl of the reaction mixture is then transferred into a 96-well P81 filter plate (Millipore) and incubated at room temperature for 5 min. The filter plate is then washed 4 times with 200 μl 75 mM phosphoric acid per well on a plate vacuum manifold (Millipore) and dried in an oven for 2-3 h. Packard MicroScint ‘0’ (30 μl) is then added to each well, the plate is mixed for 30 min and subjected to liquid scintillation counting on a Packard TopCount.

[Interpretation]
%Control=(X−B)/(Tot−B)×100
%Inhibition=100−% Control
X=cpm of the test compound wells
B=cpm of wells without enzyme
Tot=cpm of wells with DMSO vehicle only

The efficacy of the compounds in Table B against kinases is shown in Table C. (The activity is presented as +, ++, or +++ representing active, more active and very active based on assays conducted at typically 1-100 μM.)

TABLE C

| | MAPKAP-K2 |
|---|---|
| Compound No | activity |
| 1 | ++ |
| 2 | +++ |
| 3 | +++ |
| 4 | ++ |
| 5 | +++ |
| 6 | +++ |
| 7 | ++ |
| 8 | ++ |
| 9 | +++ |
| 10 | +++ |
| 11 | +++ |
| 12 | +++ |
| 13 | +++ |
| 14 | ++ |
| 15 | +++ |
| 16 | +++ |
| 17 | +++ |
| 18 | + |
| 19 | +++ |
| 20 | +++ |
| 21 | +++ |
| 22 | +++ |
| 23 | + |
| 24 | + |
| 25 | + |
| 26 | +++ |
| 27 | + |
| 28 | + |
| 29 | ++ |
| 30 | ++ |
| 31 | + |
| 32 | +++ |
| 33 | ++ |
| 34 | ++ |
| 35 | +++ |
| 36 | +++ |
| 37 | +++ |
| 38 | + |
| 39 | ++ |
| 40 | +++ |
| 41 | +++ |
| 42 | +++ |
| 43 | +++ |
| 44 | +++ |
| 45 | ++ |
| 46 | +++ |
| 47 | ++ |
| 48 | ++ |
| 49 | + |
| 50 | + |
| 51 | +++ |
| 52 | +++ |
| 53 | ++ |
| 54 | ++ |
| 55 | ++ |
| 56 | ++ |
| 57 | + |
| 58 | +++ |
| 59 | +++ |
| 60 | +++ |
| 61 | + |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | ++ |
| 72 | ++ |
| 73 | ++ |
| 74 | +++ |
| 75 | +++ |
| 76 | +++ |
| 77 | +++ |
| 78 | + |
| 79 | ++ |
| 80 | + |
| 81 | + |
| 82 | + |
| 83 | +++ |
| 84 | +++ |
| 85 | +++ |
| 86 | ++ |
| 87 | +++ |
| 88 | ++ |
| 89 | +++ |
| 90 | ++ |
| 91 | +++ |
| 92 | +++ |
| 93 | ++ |
| 94 | + |
| 95 | + |
| 96 | ++ |
| 97 | +++ |
| 98 | +++ |
| 99 | +++ |
| 100 | ++ |
| 101 | +++ |
| 102 | +++ |
| 103 | ++ |
| 104 | +++ |
| 105 | +++ |
| 106 | +++ |
| 107 | +++ |
| 108 | +++ |
| 109 | +++ |
| 110 | ++ |
| 111 | ++ |
| 112 | +++ |
| 113 | +++ |
| 114 | ++ |
| 115 | +++ |
| 116 | + |
| 117 | + |
| 118 | ++ |
| 119 | ++ |
| 120 | + |
| 121 | ++ |
| 122 | ++ |
| 123 | ++ |
| 124 | ++ |
| 125 | ++ |
| 126 | + |
| 127 | ++ |
| 128 | ++ |
| 129 | ++ |
| 130 | + |
| 131 | ++ |
| 132 | ++ |
| 133 | + |
| 134 | ++ |
| 135 | ++ |
| 136 | +++ |
| 137 | + |
| 138 | + |
| 139 | +++ |
| 140 | +++ |
| 141 | +++ |

### INDUSTRIAL APPLICABILITY

The Pyrazolo[1,5-a]pyrimidine derivatives represented by formula I and their pharmaceutically acceptable salts exhibit excellent kinase inhibiting activity particularly MAPKAP-K2 inhibiting activity). Drugs comprising the compounds as effective ingredients are therefore expected to be useful as therapeutic or prophylactic agents for a protein kinase mediated disorder in which kinase is implicated, such as such as inflammatory disease, autoimmune disease, destructive bone disorder, cancer and/or tumour growth.

1. A use of a compound of formula (I): 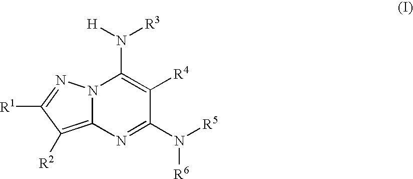
wherein R¹ is hydrogen
R² is hydrogen
R³ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl, optionally substituted heteroarylalkyl, optionally substituted arylalkenyl, optionally substituted heteroarylalkenyl, optionally substituted arylalkynyl, or optionally substituted heteroarylalkynyl;
R⁴ is hydrogen;
R⁵ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl, optionally substituted heteroarylalkyl, optionally substituted arylalkenyl, optionally substituted heteroarylalkenyl, optionally substituted arylalkynyl, or optionally substituted heteroarylalkynyl, optionally substituted heterocyclyl or optionally substituted heterocyclylalkyl;
R is hydrogen, C1-C8 optionally substituted alkyl, C2-C8 optionally substituted alkenyl, C2-C8 optionally substituted alkynyl or C3-C8 optionally substituted cycloalkyl; or R⁵ and R⁶ together may be taken together with the nitrogen to which they are attached to form a mono or bicyclic heterocycle with 5-7 members in each ring and optionally containing, in addition to the nitrogen, one or two additional heteroatoms selected from N, O and S, the said mono or bicyclic heterocycle may optionally be substituted with one or more substituents;
or pharmaceutically acceptable salts, or other pharmaceutically acceptable biohydrolyzable derivatives thereof, including esters, amides, carbamates, carbonates, ureides, solvates, hydrates, affinity reagents or prodrugs thereof, in the manufacture of a medicament for use in inhibiting protein kinases.

2. The use as claimed in claim 1, wherein R³ is C1-C8 optionally substituted alkyl, C2-C8 optionally substituted alkenyl, C2-C8 optionally substituted alkynyl, C3-C8 optionally substituted cycloalkyl, optionally substituted aryl, optionally substituted heteroaryl, optionally substituted arylalkyl or optionally substituted heteroarylalkyl.

3. The use as claimed in claim 2, wherein R³ is C2-C8 optionally substituted alkenyl, optionally substituted aryl or optionally substituted arylalkyl.

4. The use as claimed in any one of claims 1 to 3, wherein R⁵ is C1-C8 optionally substituted alkyl, C2-C8 optionally substituted alkenyl, C2-C8 optionally substituted alkynyl, C3-C8 optionally substituted cycloalkyl, optionally substituted heterocyclyl or optionally substituted heterocyclylalkyl.

5. The use as claimed in claim 4, wherein R⁵ is C3-C8 cycloalkyl substituted by NHR⁷, wherein R⁷ is optionally substituted heterocyclyl or optionally substituted heterocyclylalkyl.

6. The use as claimed in any one of claims 1 to 5, wherein R⁶ is hydrogen or C1-C8 optionally substituted alkyl.

7. The use as claimed in claim 6, wherein R⁶ is hydrogen.

8. The use as claimed in any one of claims 1 to 7, wherein the medicament is for use as an inhibitor of MAPKAP-K2.

9. The use as claimed in claim 8, wherein the medicament is for use in the prevention or treatment of a MAPKAP-K2-mediated disorder.

10. The use as claimed in claim 9, wherein the MAPKAP-K2 mediated disorder is a neurological disorder (including dementia), an inflammatory disease, a disorder linked to apoptosis, particularly neuronal apoptosis, stroke, sepsis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation.

11. The use as claimed in claim 10, wherein the disorder is a neurodegenerative disorder.

12. The use as claimed in claim 11, wherein the neurodegenerative disorder is dementia, Alzheimer's disease, Parkinson's disease, Amyotrophic Lateral Sclerosis, Huntington's disease, senile chorea, Sydenham's chorea, hypoglycemia, head and spinal cord trauma including traumatic head injury, acute and chronic pain, epilepsy and seizures, olivopontocerebellar dementia, neuronal cell death, hypoxia-related neurodegeneration, acute hypoxia, glutamate toxicity including glutamate neurotoxicity, cerebral ischemia, dementia linked to meningitis and/or neurosis, cerebrovascular dementia, or dementia in an HIV-infected patient.

13. The use as claimed in claim 10, wherein the disorder results from inflammation.

14. The use as claimed in claim 13, wherein the disorder is inflammatory bowel disorder, bronchitis, asthma, acute pancreatitis, chronic pancreatitis, allergies of various types or Alzheimer's disease.

15. The use as claimed in claim 10, wherein the disorder is an autoimmune disease.

16. The use as claimed in claim 15, wherein the autoimmune disease is rheumatoid arthritis, systemic lupus erythematosus, glomerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.

17. A method of treating or preventing a MAPKAP-K2-mediated disorder, which comprises administering to said individual at least one compound as defined in any one of claims 1 to 7 or the composition defined in claim 8 or claim 9.

18. The method as claimed in claim 17, wherein the MAPKAP-K2 mediated disorder is a neurological disorder (including dementia), an inflammatory disease, a disorder linked to apoptosis, particularly neuronal apoptosis, stroke, sepsis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation.

19. The method as claimed in claim 18, wherein the disorder is a neurodegenerative disorder.

20. The method as claimed in claim 19, wherein the neurodegenerative disorder is dementia, Alzheimer's disease, Parkinson's disease, Amyotrophic Lateral Sclerosis, Huntington's disease, senile chorea, Sydenham's chorea, hypoglycemia, head and spinal cord trauma including traumatic head injury, acute and chronic pain, epilepsy and seizures, olivopontocerebellar dementia, neuronal cell death, hypoxia-related neurodegeneration, acute hypoxia, glutamate toxicity including glutamate neurotoxicity, cerebral ischemia, dementia linked to meningitis and/or neurosis, cerebrovascular dementia, or dementia in an HIV-infected patient.

21. The method as claimed in claim 18, wherein the disorder results from inflammation.

22. The method as claimed in claim 21, wherein the disorder is inflammatory bowel disorder, bronchitis, asthma, acute pancreatitis, chronic pancreatitis, allergies of various types or Alzheimer's disease.

23. The method as claimed in claim 18, wherein the disorder is an autoimmune disease.

24. The method as claimed in claim 23, wherein the autoimmune disease is rheumatoid arthritis, systemic lupus erythematosus, glomerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.

25. The method as claimed in any one of claims 18 to 24 wherein one or more active agents is/are administered to the individual simultaneously, subsequently or sequentially to administering the compound.

26. A method for determining the activity of the compounds as defined in any one of claims 1 to 7, comprising providing a system for assaying the activity and assaying the activity of a compound as defined in any of claims 1 to 7.

27. The method as claimed in claim 26 wherein the assay is for the protein kinase inhibiting activity of the compound.

28. A method of inhibiting the activity or function of a protein kinase, which comprises exposing a protein kinase to a compound as defined in any of claims 1 to 7.

29. A method of inhibiting the activity or function of MAPKAP-K2, which comprises exposing MAPKAP-K2 to a compound as defined in any of claims 1 to 7.

30. The method as claimed in claim 29, which is performed in a research model, in vitro, in silico, or in vivo.


SEQUENCE LISTING (2 sequences)

SEQ ID NO: 1 - protein, 10 aa, Homo sapiens
  misc_feature (1)..(2): Xaa can be any naturally occurring amino acid
XXXXRXXSXX

SEQ ID NO: 2 - protein, 10 aa, Artificial Sequence
  Chemically-synthesized MAPKAP-K2 substrate
KKLNRTLSVA